(19) United States
(12) Patent Application Publication
(10) Pub. No.: US 2019/0290685 A1
(43) Pub. Date: Sep. 26, 2019

(54) COMPOSITIONS AND ASSOCIATED METHODS OF MESOPOROUS NANOPARTICLES COMPRISING PLATINUM-ACRIDINE MOLECULES

(71) Applicant: Wake Forest University, Winston-Salem, NC (US)

(72) Inventors: Ulrich Bierbach, Winston-Salem, NC (US); Ye Zheng, Houston, TX (US); Ravi Singh, Gastonia, NC (US)

(21) Appl. No.: 16/345,264

(22) PCT Filed: Oct. 27, 2017
(86) PCT No.: PCT/US17/58749

§ 371 Date: Apr. 26, 2019

Related U.S. Application Data
(60) Provisional application No. 62/414,335, filed on Oct. 28, 2016.

(51) Int. Cl. A61K 33/243; A61K 9/51; A61P 35/00

(52) U.S. Cl. CPC A61K 33/243; B82Y 5/00; A61P 35/00; A61K 9/5146

(57) ABSTRACT

Large-pore mesoporous silica nanoparticles (MSN) were prepared and functionalized to serve as a robust and biocompatible delivery platform for platinum-acridine (PA) anticancer agents. The material showed a high loading capacity for the dicationic, hydrophilic hybrid agent [PtCl(en)(N-[acridin-9-ylaminoethyl]-N-methylropionamidine)] dinitrate salt (P1 Al) and virtually complete retention of payload at neutral pH in a high-chloride buffer. In acidic media mimicking the pH inside the cells' lysosomes, rapid, burst-like release of P1 A1 from the nanoparticles is observed. Coating of the materials in phospholipid bilayers resulted in nanoparticles with greatly improved colloidal stability. The lipid and carboxylate- modified nanoparticles containing 40 wt. % drug caused S phase arrest and inhibited cell proliferation in pancreatic cancer cells at submicromolar concentrations similar to carrier-free P1A1. One feature of the nanoparticle-delivered P1A1 was that the payload did not escape from the acidified lysosomal vesicles into the cytoplasm, but was shuttled to the nuclear membrane and released into the nucleus.

### CROSS REFERENCE TO RELATED APPLICATION

This application claims priority to U.S. Provisional Application No. 62/414,335 filed on Oct. 28, 2016. The content of the application is incorporated herein by reference in its entirety.

### STATEMENT REGARDING FEDERALLY FUNDED RESEARCH

The research leading to the present application was supported in part, by National Institute of Health Grant No. R01CA101880. Accordingly, the U.S. Government has certain rights in this invention.

### FIELD OF THE INVENTION

The present application relates to pharmaceutical compositions comprising mesoporous nanoparticles in combination with a platinum-acridine agent. The mesoporous nanoparticles help deliver the platinum-acridine molecule to the site where cancer is found and release the molecule in a controlled manner

### BACKGROUND OF THE INVENTION

Many potent cytotoxins in use as cancer chemotherapies, including platinum-based agents, suffer from poor drug-like properties, which often lead to limited efficacy and severe systemic toxicities. A major drawback of most oncology drugs, such as genome (DNA)-targeted agents, is their lack of target selectivity, resulting in damage to healthy tissues and undesired side effects (H. A. Burris, 3rd, Oncogene 2009, 28 Suppl. 1, S4-13). Several strategies are being pursued to improve the safety and pharmacokinetics of anticancer drugs, including prodrug designs, attachment to tumor-targeted carrier molecules, and encapsulation of payload in nano-sized particles (T. C. Johnstone, et al., Chem. Rev. 2016, 116, 3436-3486; S. Mitragotri and J. Lahann, Adv. Mater. 2012, 24, 3717-3723; N. Larson and H. Ghandehari, Chem. Mat. 2012, 24, 840-853; S. C. Alley et al., Curr. Opin. Chem. Biol. 2010, 14, 529-537; S. Ding and U. Bierbach, Future Med. Chem. 2015, 7, 911-927). Major advantages of the latter delivery mode, such as polyethylene glycol-modified (“PEGylated”) stealth liposomes, (Y. Barenholz, J. Control. Release 2012, 160, 117-134; b) G. P. Stathopoulos, Anticancer Drugs 2010, 21, 732-736) include prolonged circulation times and improved bioavailability in diseased tissues via selective passive accumulation (I. K. Kwon, et al., J. Control. Release 2012, 164, 108-114) (enhanced permeability and retention, EPR). Several strategies have been validated for release of payloads from nanocarriers and activation of drugs in tumor tissue, such as physical and chemical-physiological stimuli (light, heat, pH, hypoxia, ion gradients), as well as enzymatically degradable linkers and polymers (K. Cho, et al., Clin. Cancer Res. 2008, 14, 1310-1316; S. Mura, J. Nicolas and P. Couvreur, Nat. Mater. 2013, 12, 991-1003).

Platinum-acridines are a class of DNA-targeted hybrid agents, which have shown promising activity in solid tumor models.

### SUMMARY OF THE INVENTION

The present invention relates to pharmaceutical compositions containing nanoparticles that have been specially designed to deliver platinum acridines to cancer cells with improved pharmacological properties and reduced systemic toxicity. The inert mesoporous silica nanoparticles and the large surface area of the pores made them an ideal vehicle for allowing the nanoparticles to be filled with a drug or a cytotoxin. The nanoparticles can be used in a manner that allows them to be taken up by certain biological cells through endocytosis, depending on the functionalities that are attached to the outside of the spheres. Some types of cancer tissues can be targeted more efficiently by the particles relative to healthy tissues allowing researchers to selectively deliver drugs or cytotoxins to cancer cells.

In this invention, large-pore mesoporous silica nanoparticles (MSN) were synthesized using conventional hexadecyltrimethylammonium bromide (aka cetyltrimethylammonium bromide or CTAB) as soft template. Large pore sizes (>6.5 nm) and high surface areas (>700 m²/g) were achieved with a new synthetic strategy using a swelling agent/co-solvent mixture containing n-decane and dimethylformamide (DMF) Silanes with different molecular lengths of polyethylene glycol (PEG) were used to enhance the aqueous dispersibility of the materials in biologically relevant media. Alternatively, MSN were coated with lipid films consisting of zwitterionic and cationic lipids, which resulted in excellent colloidal stability. The inner surface of MSN was further functionalized with carboxylic groups. Various characterization methods were used including Fourier-transform infrared spectroscopy (FTIR), small-angle X-ray scattering (SAXS), thermogravimetric analysis (TGA), isothermal gas adsorption, transmission electron microscopy (TEM), and dynamic light scattering (DLS). The uptake of dicationic platinum-acridines into, and release from, selected materials was studied in media mimicking physiological conditions in plasma and cells. The results suggest that optimized MSN-based materials may have utility as carriers for the safe delivery of platinum-acridines to target tissues. Also provided is a method of treating cancer comprising administering to an individual in need thereof the pharmaceutical composition describe herein.

The present invention, in one embodiment, provides a pharmaceutical composition comprising a compound of formula I and a silica mesoporous nanoparticle;

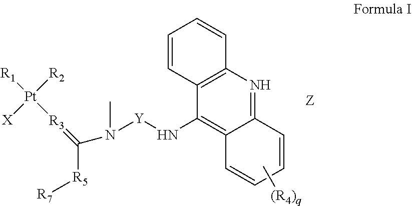

wherein X is halo, —OC(O)R₉, nitrate, H₂O or sulfate;
R₁ and R₂ are amino groups or together with the platinum atom to which they are attached, R₁ and R₂ form the ring —NH₂—(CH₂)ᵥ—NH₂— wherein v is 1, 2, or 3;

R₃ is —N(R₆)—, wherein R₆ is hydrogen or C₁-C₆alkyl;

each R₄ is independently an amino, a nitro, —NHC(O)(R₁₀), —C(O)NHR₁₀, or halo;

R₁₀ is hydrogen, C₁₋₆ alkyl, phenyl, naphthyl, C₃₋₆ cycloalkyl, norbornyl, or adamantyl;

q is 0, 1, or 2;

R₅ is a direct bond, —NH— or C₁-C₆alkylene;

or R₅ and X together with the atoms to which they are attached form a 6- or 7-membered ring, wherein said 6- or 7-membered ring contains a linking group —C(O)O— or —OC(O)—;

R₇ is hydrogen, methyl, or —C(O)O—R₈; wherein

R₈ is hydrogen, C₁₋₆ alkyl, phenyl, naphthyl, C₃₋₆ cycloalkyl, norbornyl, or adamantyl, a natural or unnatural amino acid or a peptide;

R₉ is hydrogen, C₁₋₆ alkyl, phenyl, naphthyl, C₃₋₆ cycloalkyl, norbornyl, or adamantyl;

Y is C₁-C₆alkyl; and

Z is one or more counterions sufficient to balance the charge of the compound.

In another embodiment, the present invention provides a pharmaceutical composition wherein the silica mesoporous nanoparticle has a pore size of about 6.5 nm or greater.

In another embodiment, the present invention provides a pharmaceutical composition wherein the silica mesoporous nanoparticle has a surface area of about at least 700 m²/g.

In another embodiment, the present invention provides a pharmaceutical composition wherein the compound is [PtCl(en)(N-[acridin-9-ylaminoethyl]-N-methylpropionamidine)] dinitrate salt.

In another embodiment, the present invention provides a pharmaceutical composition wherein the silica mesoporous nanoparticle is made by reacting cetyltrimethylammonium bromide with tetraethylorthosilicate.

In another embodiment, the present invention provides a pharmaceutical composition wherein the silica mesoporous nanoparticle further comprises polyethylene glycol.

In another embodiment, the present invention provides a pharmaceutical composition wherein the silica mesoporous nanoparticle further comprises a lipid bilayer.

In another embodiment, the present invention provides a pharmaceutical composition wherein the lipid bilayer is a phospholipid bilayer.

In another embodiment, the present invention provides a pharmaceutical composition wherein the phospholipid bilayer comprises one or more of 1,2-dipalmitoyl-sn-glycero-3-phosphocholine, 1,2-dioleoyl-3-trimethylammoniumpropane, 1,2-dipalmitoyl-sn-glycero-3-phosphoethanolamine-N-[-[methoxyethyleneglycol)-2000, 1,2-Distearoyl-sn-glycero-3-phosphoethanolamine (DSPE)-conjugated and fluorescein-labeled polyethyleneglycol, and cholesterol.

In another embodiment, the present invention provides a pharmaceutical composition wherein the compound is [PtCl(en)(N-[acridin-9-ylaminoethyl]-N-methylpropionamidine)] dinitrate salt and the silica mesoporous nanoparticle comprises a reaction product of cetyltrimethylammonium bromide with tetraethylorthosilicate.

In another embodiment, the present invention provides a pharmaceutical composition wherein the reaction product of cetyltrimethylammonium bromide with tetraethylorthosilicate is derived from a reaction done in basic aqueous solution comprising NaOH in the presence of dimethylformamide and n-decane.

In another embodiment, the present invention provides a method of treating cancer comprising administering to an individual in need thereof a pharmaceutical composition comprising a compound of formula I and a silica mesoporous nanoparticle.

In another embodiment, the present invention provides a method of treating pancreatic cancer comprising administering to an individual in need thereof a pharmaceutical composition comprising a compound of formula I and a silica mesoporous nanoparticle.

In another embodiment, the present invention provides a method of treating cancer comprising administering to an individual in need thereof a pharmaceutical composition comprising a compound of formula I and a silica mesoporous nanoparticle, wherein the silica mesoporous nanoparticle of the pharmaceutical composition has a pore size of about 6.5 nm or greater.

In another embodiment, the present invention provides a method of treating cancer comprising administering to an individual in need thereof a pharmaceutical composition comprising a compound of formula I and a silica mesoporous nanoparticle, wherein the silica mesoporous nanoparticle of the pharmaceutical composition has a surface area of about at least 700 m²/g.

In another embodiment, the present invention provides a method of treating cancer comprising administering to an individual in need thereof a pharmaceutical composition comprising a compound of formula I and a silica mesoporous nanoparticle, wherein the silica mesoporous nanoparticle delivers the compound to the nucleus of a cancer cell.

In another embodiment, the present invention provides a method of treating cancer comprising administering to an individual in need thereof a pharmaceutical composition comprising a compound of formula I and a silica mesoporous nanoparticle, wherein the silica mesoporous nanoparticle is charged with at least about 40 wt.% of the compound.

In another embodiment, the present invention provides a method of treating cancer comprising administering to an individual in need thereof a pharmaceutical composition comprising a compound of formula I and a silica mesoporous nanoparticle, wherein the pharmaceutical composition remains intact at a pH of about 7 and the compound is released from the pharmaceutical composition at a pH of between about 4 and 5.

In another embodiment, the present invention provides a method of treating cancer comprising administering to an individual in need thereof a pharmaceutical composition comprising a compound of formula I and a silica mesoporous nanoparticle, wherein the compound is[PtCl(en)(N-[acridin-9-ylaminoethyl]-N-methylpropionamidine)]dinitrate salt.

In another embodiment, the present invention provides a method of treating cancer comprising administering to an individual in need thereof a pharmaceutical composition comprising a compound of formula I and a silica mesoporous nanoparticle, wherein the silica mesoporous nanoparticle is made by a process comprising reacting cetyltrimethylammonium bromide with tetraethylorthosilicate in n-decane and dimethylformamide

### BRIEF DESCRIPTION OF THE FIGURES

FIGS. 1A and 1B illustrate the characterization of small-pore (MSN_{SP}, MCM-41) and large-pore (MSN_{LP}) mesoporous nanoparticles. FIG. 1A illustrates transmission electron microscopy (TEM) images of MSN_{SP} (left) and MSN_{LP} (right). FIG. 1B illustrates SAXS diffraction patterns, nitrogen physisorption isotherms, and computed NLDFT pore size distribution (PSD) (left to right) for MSN_{SP} and MSN_{LP}. Note the change in the adsorption/desorption isotherms from Type IV(b) to Type IV(a) (according to current IUPAC classification) and the pronounced hysteresis loop due to capillary condensation in MSN_{LP}.

FIG. 2A illustrates a comparison of PA loading capacity of MSN_{SP}-COOH and MSN_{LP}-COOH.

FIG. 2B illustrates TEM bright-field image of P1A1@MSN_{LP}-COOH (12 wt. % P1A1).

FIG. 2C illustrates STEM high-angle annular dark-field (HAADF) image and EDS mapping for Si and Pt for P1A1@MSN_{LP}-COOH.

FIG. 3 illustrates the preparation of P1A1@MSN_{LP}-COOH-PEG (route I) and P1A1@MSN_{LP}-COOH-LIP (route II) nanoparticles.

FIGS. 4A and 4B illustrate the composition of multi-component nanoparticles assessed by TGA. FIG. 4C illustrates the effect of MSN pore size and PEG chain length on the ability of the materials to adsorb/bind P1A1. The measured weight losses for each level of modification in FIG. 4A are 7% for MSN_{LP}, 14% for MSN_{LP}-PEG₅ₖ, and 24% for MSN_{LP}-COOH, which is in excellent agreement with the cumulative weight loss of 46% observed in MSN_{LP}-COOH-PEG₅ₖ. Error bars in FIG. 4C represent±standard deviations for quadruplicate samples.

FIGS. 5A, 5B, 5C, 5D, 5E and 5F illustrate characterization of lipid-coated PA-free and PA-containing MSN materials wherein FIG. 5A illustrates TGA and FIG. 5B illustrates FT-IR analysis confirming composition of the multi-component systems. FIG. 5C illustrates TEM images of UA-stained lipid bilayer-coated MSN_{LP}-COOH-LIP₂₀ (left) and P1A1@MSN_{LP}-COOH-LIP₂₀ (right). FIG. 5D illustrates HAADF image and EDS mapping for Si, C, P, and Pt in P1A1@MSN_{LP}-COOH-LIP₂₀ (12 wt. % P1A1). FIG. 5E illustrates DLS data for P1A1@MSN_{LP}-COOH-LIP₂₀ acquired in water (hydrodynamic diameter 250 nm, polydispersity index 0.127), and in PBS after 30 min (h. d. 240 nm, PDI 0.091) and 60 min (h. d. 247 nm, PDI 0.097; blue trace). FIG. 5F illustrates spectrophotometric monitoring of payload release at 37° C. from P1A1@MSN_{LP}-COOH and P1A1@MSN_{LP}-COOH-LIP₂₀ in different buffers. Error bars represent±standard deviations for 4 samples.

FIGS. 6A and 6B illustrate the results of cell studies wherein FIG. 6A illustrates PANC-1 and BxPC3 cells treated with P1A1 [0-200 μM], equivalent P1A1@MSN_{LP}-COOH-LIP₁₀, P1A1@MSN_{LP}-COOH-LIP₂₀ (40 wt. % P1A1), or control drug-free carrier material for 72 h in quadruplicate. FIG. 6B illustrates BXPC3 cells treated as enumerated herein at concentrations ranging from 0-2000 nM. Results of the flow cytometry analysis and cell cycle distribution are shown.

FIGS. 7A, 7B and 7C illustrate confocal fluorescence microscopy images (single confocal planes) of BxPC3 pancreatic cancer cells showing uptake and subcellular distribution of FIG. 7A illustrates P1A1 and FIG. 7B illustrates P1A1@MSN_{LP}-COOH-LIP₁₀-FITC. FIG. 7C illustrates colocalization image capture after treatment of BxPC3 with P1A1@MSN_{LP}-COOH-LIP₁₀-FITC.

### DETAILED DESCRIPTION

While the following text may reference or exemplify specific embodiments of a large-pore mesoporous silica nanoparticle with a compound or use in a method of treating a disease or condition, it is not intended to limit the scope of the compound or method to such particular reference or examples. Various modifications may be made by those skilled in the art, in view of practical and economic considerations, such as the substitutions of the compound and the amount or administration of the compound for treating or preventing a disease or condition.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “alkyl” as used herein refers to monovalent saturated alkane radical groups particularly having up to about 18 carbon atoms, more particularly as a lower alkyl, from 1 to 8 carbon atoms and still more particularly, from 1 to 6 carbon atoms. The hydrocarbon chain may be either straight-chained or branched. The term “C₁-C₆ alkyl” refers to alkyl groups having 1, 2, 3, 4, 5, or 6 carbon atoms.

The term “alkylene” as used herein refers to bi-valent saturated alkane radical groups. For example, a C₁-C₂alkylene can be —CH₂— or —CH₂CH₂—.

The term “pharmaceutical composition” refers to a mixture of a compound disclosed herein with other chemical components, such as diluents or additional carriers. The pharmaceutical composition facilitates administration of the compound to an organism. Multiple techniques of administering a pharmaceutical composition exist in the art including, but not limited to, oral, injection, aerosol, parenteral, and topical administration. In some embodiments, pharmaceutically acceptable salts of the compounds disclosed herein are provided.

The term “subject” encompasses any animal, but preferably a mammal, e.g., human, non-human primate, a dog, a cat, a horse, a cow, or a rodent. More preferably, the subject is a human

The term “carrier” refers to a chemical compound that facilitates the incorporation of a compound into cells or tissues.

The term “diluent” refers to chemical compounds diluted in water that will dissolve the composition of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound. As used herein, an “excipient” refers to an inert substance that is added to a composition to provide, without limitation, bulk, consistency, stability, binding ability, lubrication, disintegrating ability, etc., to the composition. A “diluent” is a type of excipient.

The term “physiologically acceptable” or “pharmaceutically acceptable” refers to a carrier or diluent that does not abrogate the biological activity and properties of the compound.

The term “therapeutically effective amount” refers to an amount of a compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The term “treating” or “treatment” of any disease or condition refers, in some embodiments, to ameliorating the disease or disorder (i.e., arresting or reducing the development of the disease or at least one of the clinical symptoms thereof). In some embodiments “treating” or “treatment” refers to ameliorating at least one physical parameter, which may not be discernible by the subject. In some embodiments, “treating” or “treatment” refers to modulating the disease or disorder, either physically, (e.g., stabilization of a discernible symptom), physiologically, (e.g., stabilization of a physical parameter), or both. In some embodiments, “treating” or “treatment” refers to delaying the onset of the disease or disorder, or even preventing the same. “Prophylactic treatment” is to be construed as any mode of treatment that is used to prevent progression of the disease or is used for precautionary purpose for persons at risk of developing the condition.

The present invention relates to large-pore mesoporous silica nanoparticles (MSN) that have been prepared and functionalized to serve as a highly robust and biocompatible delivery platform for platinum-acridine (PA) anticancer agents. The mesoporous silica nanoparticles comprise a high loading capacity for platinum-acridine anticancer agents such as the dicationic, hydrophilic hybrid agent [PtCl(en)(N-[acridin-9-ylaminoethyl]-N-methylpropionamidine)] dinitrate salt (P1A1) and virtually complete retention of payload at neutral pH in a high-chloride buffer. In acidic media mimicking the pH inside the cells' lysosomes (pH of about 4 to 5), rapid, burst-like release of P1A1 from the nanoparticles is observed. Additionally, the coating of the materials in phospholipid bilayers resulted in nanoparticles with greatly improved colloidal stability.

Platinum-acridines (PAs) represented by the prototypical agent [PtCl(en)(N-[acridin-9-ylaminoethyl]-N-methylpropion-amidine)] dinitrate salt (en=ethane-1,2-diamine) (see Formula A below) are a class of highly cytotoxic DNA-targeted hybrid agents (A. J. Pickard, et al., Chem. Eur. J. 2014, 20, 16174-16187; J. Suryadi and U. Bierbach, Chem. Eur. J. 2012, 18, 12926-12934). PAs target genomic DNA by a dual mechanism that involves monofunctional platination of nucleobases and intercalation of the protonated 9-aminoacridine moiety between adjacent base pairs in DNA. Id. P1A1 and its newer derivatives show potent cell kill in several solid tumor models at the lowest (nanomolar) inhibitory concentrations observed for platinum-containing agents to date (S. Ding, et al., Chem. Eur. J. 2014, 20, 16164-16173). The most active, newer PA derivatives show 1000-fold higher cytotoxicity in vitro than the clinical drug cisplatin.

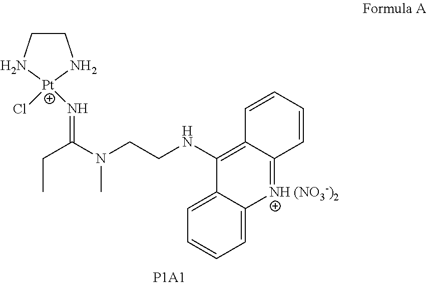

While able to slow tumor growth in mouse xenograft models, PAs are also very toxic, leading to significant weight loss in treated test animals Without being bound by theory, this is most likely due to damage to the kidneys (Z. Ma, et al., J. Med. Chem. 2008, 51, 7574-7580). Thus, one major challenge with PAs is their safety when applied in vivo. Unlike other mechanistically related oncology drugs such as cisplatin and doxorubicin, PAs exist as highly water-soluble 2+ charged cations. Because of their pronounced hydrophilicity, PAs are poorly absorbed from circulation into target tissues and prone to rapid excretion via renal clearance. Thus, in one embodiment of the present invention, the present invention deals with developing PAs into a clinically viable treatment option that improves their biodistribution and delivers them efficiently and safely to diseased tissue.

Thus, in a variation, the present invention relates to tailoring a carrier platform to the chemical requirements of a specific pharmacophore. For PAs, as well as other highly cytotoxic agents suffering from unfavorable absorption, distribution, metabolism, and excretion (ADME) properties the ideal delivery vehicle has one or more of the following properties (i) be chemically robust in circulation such that the interactions between drug and carrier are strong enough to resist premature release of payload, (ii) accommodate a highly concentrated payload to allow large-bolus delivery for treating aggressive cancers, (iii) increase accumulation in tumor tissue, and (iv) enable triggered payload-release after entering cancer cells. In one embodiment, the present invention relates to developing large-pore, carboxylate-modified mesoporous silica nanoparticles (MSN) (P. Yang, et al., Chem. Soc. Rev. 2013, 41, 3679-3698) in combination with surface PEGylation or a PEG-modified lipid bilayer (C. E. Ashley, et al., Nat. Mater. 2011, 10, 389-397) that provides an ideal vehicle for PA payloads. Mechanisms of controlled nanoparticle delivery and release of drug payload from MSN-based materials I. I. Slowing, et al., Adv. Funct. Mater. 2007, 17, 1225-1236; D. Arcos and M. Vallet-Regí, Acta Mater. 2013, 61, 890-911) employ mechanisms that involve redox chemistry (C.-Y. Lai, et al., J. Am. Chem. Soc. 2003, 125, 4451-4459), proton gradients (L. Han, O. Terasaki and S. Che, J. Mat. Chem. 2011, 21, 11033-11039), magnetism (S. Huang, et al., J. Colloid. Interface Sci. 2012, 376, 312-321), electromagnetic radiation (N. K. Mal, et al., Nature 2003, 421, 350-353; M. Bathfield, et al., Chem. Mater. 2016, 28, 3374-3384), and activation by enzymes (A. Agostini, et al., Chemistry Open 2012, 1, 17-20).

The negative surface charge of MSN and the dicationic payload of the PAs are electrostatically highly compatible, and the large pore diameter of the MSNs that have been developed allow deep penetration of PAs into the material. In addition to charge-driven adsorption, coordinative binding of platinum to grafted carboxylate groups (J. Gu, et al., J. Phys. Chem. Lett. 2010, 1, 3446-3450; C.-H. Lin, et al., Int. J. Pharm. 2012, 429, 138-147) via substitution of the labile chloro ligand in P1A1 further enhances payload uptake and retention. Monodentate carboxylate coordination also allows triggered release of drug (PAs) via ligand exchange in cells in the low-pH environment of the lysosomes (F. Meng, et al., Mater. Today 2012, 15, 436-442).

The results presented herein suggest that the carrier system designed in this way possesses several unique features ideally suited for its use as a targeted delivery vehicle for this type of hybrid agent.

The lipid and carboxylate-modified nanoparticles containing about for example 40 wt. % drug caused S phase arrest and inhibited cell proliferation in pancreatic cancer cells at submicromolar concentrations similar to carrier-free P1A1. One feature of nanoparticle-delivered P1A1 was that the payload did not escape from the acidified lysosomal vesicles into the cytoplasm, but was shuttled to the nuclear membrane and released into the nucleus. In addition to selective delivery to diseased tissue, MSNs can also be used to protect PA from intracellular detoxification and efflux of drug and deliver PA to DNA in the nucleus in forms of cancer where this type of resistance (e.g., in types where the molecule is released in the cytoplasm) limits efficacy.

Accordingly, the large-pore mesoporous silica nanoparticles provide an ideal avenue for introducing platinum-acridine anticancer agents to the nucleus of cancer cells, meaning that the platinum-acridine anticancer agents will be delivered where they trigger cancer cell death.

In one embodiment, the PA compounds that are within the scope of the present invention are defined by Formula I.

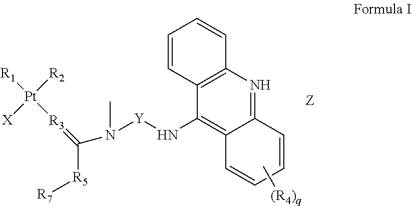

wherein X is halo, OC(O)R₉nitrate, H₂O or sulfate;

R₁ and R₂ are amino groups or together with the platinum atom to which they are attached, R₁ and R₂ form the ring —NH₂—(CH₂)ᵥ—NH₂— wherein v is 1, 2, or 3, or R₁ and R₂ together can be any of the following groups a-h or R₁ and R₂ independently can be any of i-m;

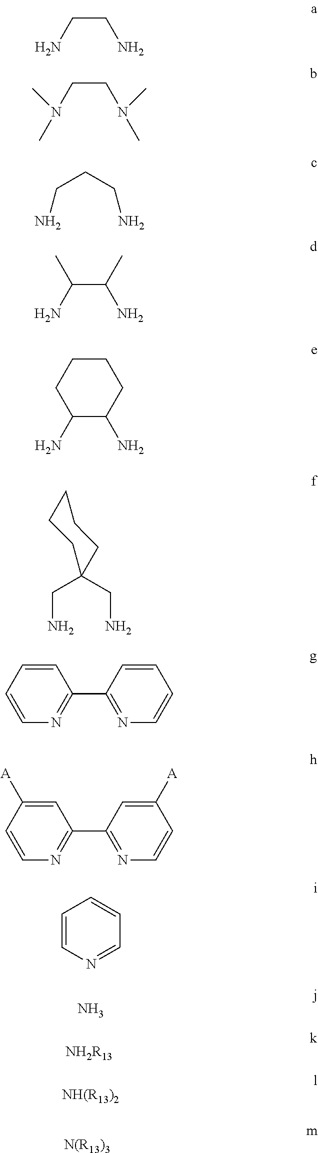

wherein A is H, —CH₃, —OCH₃, CF₃ or NO₂;
R¹³ is independently C₁-C₆alkyl;
R₃ is —N(R₆)—; wherein R₆ is hydrogen or C₁-C₆alkyl;
R₄ is independently an amino, a nitro, —NHC(O)(R₁₀), —C(O)NHR₁₀, or halo;
R₁₀ is hydrogen, C₁₋₆ alkyl, phenyl, naphthyl, C₃₋₆ cycloalkyl, norbornyl, or adamantyl;
q is 0, 1, or 2;
R₅ is a direct bond, —NH— or C₁-C₆alkylene;
or R₅ and X together with the atoms to which they are attached form a 6- or 7-membered ring, wherein said 6- or 7-membered ring contains a linking group —C(O)O— or —OC(O)—;
R₇ is hydrogen, methyl, or —C(O)O—R₈; wherein
R₈ is hydrogen, C₁₋₆ alkyl, phenyl, naphthyl, C₃₋₆ cycloalkyl, norbornyl, adamantyl, a natural or unnatural amino acid or a peptide;
R₉ is hydrogen, C₁₋₆ alkyl, phenyl, naphthyl, C₃₋₆ cycloalkyl, norbornyl, adamantyl, a natural or unnatural amino acid or a peptide;
Y is C₁-C₆alkyl; and
Z is one or more counterions sufficient to balance the charge of the compound.

In some embodiments, the PA compound is

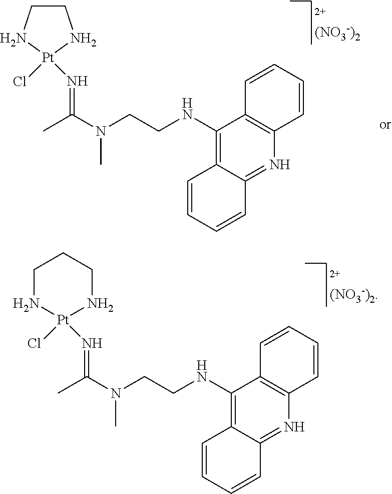

In an embodiment, the present invention discloses methods of treating cancer in an individual in need thereof by the use of a compound of Formula I.

In a variation, the compounds of the present invention can be used for treating diseases of abnormal cell growth and/or dysregulated apoptosis, such as cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, ovarian cancer, cervical cancer, colon cancer, rectal cancer, cancer of the anal region, stomach cancer, gastrointestinal (gastric, colorectal, and duodenal), chronic lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular cancer (hepatic and biliary duct), primary or secondary central nervous system tumors, primary or secondary brain tumors, Hodgkin's disease, chronic or acute leukemias, chronic myeloid leukemia, lymphocytic lymphomas, lymphoblastic leukemia, follicular lymphoma, lymphoid malignancies of T-cell or B-cell origin, melanoma, multiple myeloma, oral cancer, ovarian cancer, non-small cell lung cancer, prostate cancer, small-cell lung cancer, cancer of the kidney and ureter, renal cell carcinoma, carcinoma of the renal pelvis, neoplasms of the central nervous system, primary central nervous system lymphoma, non-Hodgkin's lymphoma, spinal axis tumors, brains stem glioma, pituitary adenoma, adrenocortical cancer, gall bladder cancer, cancer of the spleen, cholangiocarcinoma, fibrosarcoma, neuroblastoma, retinoblasitoma, or a combination thereof.

In a further variation, the compounds of the present invention can be used in methods of treating mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, ovarian cancer, cervical cancer, colon cancer, rectal cancer, cancer of the anal region, stomach cancer, gastrointestinal (gastric, colorectal, and duodenal), chronic lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular cancer (hepatic and biliary duct), primary or secondary central nervous system tumor, primary or secondary brain tumor, Hodgkin's disease, chronic or acute leukemia, chronic myeloid leukemia, lymphocytic lymphomas, lymphoblastic leukemia, follicular lymphoma, lymphoid malignancies of T-cell or B-cell origin, melanoma, multiple myeloma, oral cancer, ovarian cancer, non-small cell lung cancer, prostate cancer, small-cell lung cancer, cancer of the kidney and ureter, renal cell carcinoma, carcinoma of the renal pelvis, neoplasms of the central nervous system, primary central nervous system lymphoma, non-Hodgkin's lymphoma, spinal axis tumors, brains stem glioma, pituitary adenoma, adrenocortical cancer, gall bladder cancer, cancer of the spleen, cholangiocarcinoma, fibrosarcoma, neuroblastoma, retinoblasitoma, or a combination of one or more of the above cancers in a patient, said methods comprising administering thereto a therapeutically effective amount of a compound having formula (II).

In a further variation, the compounds of the present invention can be used for treating bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small-cell lung cancer and spleen cancer.

In a variation of the method, the cancer may alternatively be selected from the group consisting of lung cancer, genitourinal cancers, bladder cancers, testicular cancers, ovarian carcinomas, various head and neck cancers, colon cancers, various leukemias, and various lymphomas.

In another variation of the method, the variables of formula I may be any of the follows:

R₃ may be —N(R₆)—, wherein R₆ is C₁₋₆alkyl or hydrogen. In a variation, Y may be —CH₂—. In a variation, R₁ and R₂ may be amino groups or together with the platinum atom to which R₁ and R₂ are attached are —NH₂—CH₂—NH₂—. In a variation, the counter ion Z comprises NO₃. In a further variation, R₅ may be —NH— or —CH₂—. In a further variation, R₆ may be hydrogen or methyl.

In another embodiment, the present invention is directed to a pharmaceutical composition comprising the compound of Formula 1:

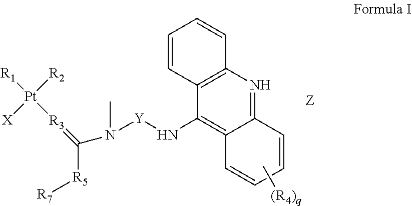

wherein X is halo, —OC(O)R₉, nitrate, H₂O or sulfate;

R₁ and R₂ are amino groups or together with the platinum atom to which they are attached, R₁ and R₂ form the ring —NH₂—(CH₂)ᵥ—NH₂— wherein v is 1, 2, or 3;
R₃ is —N(R₆)—, wherein R₆ is hydrogen or C₁-C₆alkyl;
R₄ is independently an amino, a nitro, —NHC(O)(R₁₀), —C(O)NHR₁₀, or halo;
R₁₀ is hydrogen, C₁₋₆ alkyl, phenyl, naphthyl, C₃₋₆ cycloalkyl, norbornyl, or adamantyl;
q is 0, 1, or 2;
R₅ is a direct bond, —NH— or C₁-C₆alkylene;
or R₅ and X together with the atoms to which they are attached form a 6- or 7-membered ring, wherein said 6- or 7-membered ring contains a linking group —C(O)O— or —OC(O)—;
R₇ is hydrogen, methyl, or —C(O)O—R₈; wherein
R₈ is hydrogen, C₁₋₆ alkyl, phenyl, naphthyl, C₃₋₆ cycloalkyl, norbornyl, or adamantyl, a natural or unnatural amino acid or a peptide;
R₉ is hydrogen, C₁₋₆ alkyl, phenyl, naphthyl, C₃₋₆ cycloalkyl, norbornyl, or adamantyl;
Y is C₁-C₆alkyl; and
Z is one or more counterions sufficient to balance the charge of the compound.

Thus, in an embodiment, the present invention relates to a pharmaceutical composition comprising a compound of formula I and a silica mesoporous nanoparticle;

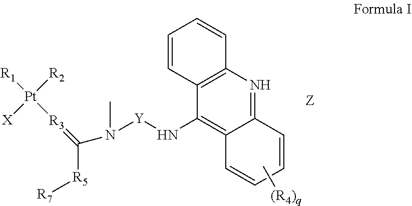

wherein X is halo, —OC(O)R₉, nitrate, H₂O or sulfate;
R₁ and R₂ are amino groups or together with the platinum atom to which they are attached, R₁ and R₂ form the ring —NH₂—(CH₂)ᵥ—NH₂— wherein v is 1, 2, or 3;

R₃ is —N(R₆)—, wherein R₆ is hydrogen or C₁-C₆alkyl;

R₄ is independently an amino, a nitro, —NHC(O)(R₁₀), —C(O)NHR₁₀, or halo;

R₁₀ is hydrogen, C₁₋₆ alkyl, phenyl, naphthyl, C₃₋₆ cycloalkyl, norbornyl, or adamantyl;

q is 0, 1, or 2;

R₅ is a direct bond, —NH— or C₁-C₆alkylene;

or R₅ and X together with the atoms to which they are attached form a 6- or 7-membered ring, wherein said 6- or 7-membered ring contains a linking group —C(O)O— or —OC(O)—;

R₇ is hydrogen, methyl, or —C(O))O—R₈; wherein

R₈ is hydrogen, C₁₋₆ alkyl, phenyl, naphthyl, C₃₋₆ cycloalkyl, norbornyl, or adamantyl, a natural or unnatural amino acid or a peptide;

R₉ is hydrogen, C₁₋₆ alkyl, phenyl, naphthyl, C₃₋₆ cycloalkyl, norbornyl, or adamantyl;

Y is C₁-C₆alkyl; and

Z is one or more counterions sufficient to balance the charge of the compound.

In some embodiments, the PA compound is

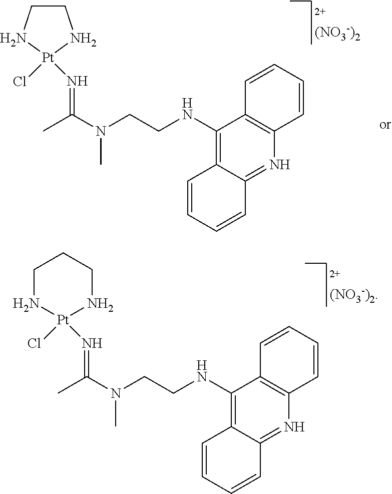

In one variation, the pharmaceutical composition comprises a silica mesoporous nanoparticle that is a silica mesoporous nanoparticle with a pore size of about 5.5 nm or greater, about 6.0 nm or greater, about 6.5 nm or greater, about 7.0 nm or greater, or about 7.5 nm or greater.

In one variation, the pharmaceutical composition comprises a silica mesoporous nanoparticle that is a silica mesoporous nanoparticle with a surface area of about at least 600 m²/g, about at least 650 m²/g, about at least 700 m²/g or about at least 800 m²/g.

In one variation, the pharmaceutical composition comprises a compound that is [PtCl(en)(N-[acridin-9-ylaminoethyl]-N-methylpropionamidine)] dinitrate salt.

In one variation, the pharmaceutical composition comprises a silica mesoporous nanoparticle that is made by reacting cetyltrimethylammonium bromide with tetraethylorthosilicate.

In an embodiment, the pharmaceutical composition comprises a silica mesoporous nanoparticle further comprises polyethylene glycol.

In one variation, the pharmaceutical composition comprises a silica mesoporous nanoparticle that further comprises a lipid bilayer. In one variation, in the lipid bilayer is a phospholipid bilayer. In one variation, the phospholipid bilayer comprises one or more of 1,2-dipalmitoyl-sn-glycero-3-phosphocholine, 1,2-dioleoyl-3-trimethylammoniumpropane, 1,2-dipalmitoyl-sn-glycero-3-phosphoethanolamine-N-[-[methoxyethyleneglycol)-2000, 1,2-Distearoyl-sn-glycero-3-phosphoethanolamine (DSPE)-conjugated and fluorescein-labeled polyethyleneglycol, cholesterol, and any mixture thereof.

In one variation, the pharmaceutical composition comprises a compound that is [PtCl(en)(N-[acridin-9-ylaminoethyl]-N-methylpropionamidine)] dinitrate salt and the silica mesoporous nanoparticle comprises a reaction product of cetyltrimethylammonium bromide with tetraethylorthosilicate.

In one variation, the pharmaceutical composition comprises a reaction product of cetyltrimethylammonium bromide with tetraethylorthosilicate that is derived from a reaction done in one or more solvents such as dimethylformamide and/or n-decane. In one embodiment, dimethyl-formamide and n-decane as solvent and cetyltrimethylammonium bromide and tetraethylorthosilicate as reactants under the specific conditions recited herein produce the special large pore large surface particles as described herein. This synthetic procedure and the conditions employed as described herein were to the inventors' knowledge unknown and thus, are completely novel as they produced MSNs that have the special properties described herein.

In one variation, the present invention relates to methods of treating cancer wherein the method comprises administering to an individual in need thereof, the pharmaceutical composition as described above.

In one embodiment, the method involves treating a cancer that is pancreatic cancer.

In one embodiment, the present invention relates to a method of using the pharmaceutical composition wherein the silica mesoporous nanoparticles have a pore size of about 6.5 nm or greater. In an embodiment, the average hydrodynamic particle diameters are between 220-250 nm and the particles have a polydispersity index (PDI) of less than about 0.1 or alternatively, about 0.1.

In an embodiment, the method uses a pharmaceutical composition that comprises a silica mesoporous nanoparticle with a surface area of about at least 700 m²/g.

In a variation, the method uses a pharmaceutical composition that comprises a silica mesoporous nanoparticle that delivers the compound to the nucleus of a cancer cell.

In one embodiment, the silica mesoporous nanoparticle is charged with at least about 30 wt. %, at least about 35 wt. %, at least about 40 wt. %, or at least about 50 wt. % of the compound (i.e., the PA compound). In one embodiment, the method uses a pharmaceutical composition wherein the combined MSN and PA remains intact (together) at a pH of about 7 and the compound is released from the silica mesoporous nanoparticle at a pH of between about 4.0 and 5.0 (about the pH of a lysosome) or alternatively, a pH of about 5.

In one variation, the method uses a compound that is [PtCl(en)(N-[acridin-9-ylaminoethyl]-N-methylpropionamidine)] dinitrate salt. In one variation of the method, the silica mesoporous nanoparticle is made by a process that reacts cetyltrimethylammonium bromide with tetraethylorthosilicate.

In a further variation, the present invention contemplates combination therapies in which the compounds of the present invention can be used in conjunction with other platinum compounds. The efficacy of this combination therapy is likely to be enhanced because of the different mechanisms and modes of action that first generation cisplatin compounds exhibit relative to the compounds of the present invention. It is also contemplated and therefore within the scope of the invention that other anti-neoplastic agents/compounds can be used in conjunction with the compounds of the present invention. The anti-neoplastic agents/compounds that can be used with the compounds of the present invention include cytotoxic compounds as well as non-cytotoxic compounds.

Examples include anti-tumor agents such as HERCEPTIN™ (trastuzumab), RITUXAN™ (rituximab), ZEVALIN™ (ibritumomab tiuxetan), LYMPHOCIDE™ (epratuzumab), GLEEVAC™ and BEXXAR™ (iodine 131 tositumomab).

Other anti-neoplastic agents/compounds that can be used in conjunction with the compounds of the present invention include anti-angiogenic compounds such as ERBITUX™ (IMC-C225), KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTIN™ or VEGF-TRAP™, and anti-VEGF receptor agents (e.g., antibodies or antigen binding regions that specifically bind thereto), EGFR inhibitory agents (e.g., antibodies or antigen binding regions that specifically bind thereto or inhibitors of the kinase domain) such as ABX-EGF (panitumumab), IRESSA™ (gefitinib), TARCEVA™ (erlotinib), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto).

Other anti-angiogenic compounds/agents that can be used in conjunction with the compounds of the present invention include Campath, IL-8, B-FGF, Tek antagonists, anti-TWEAK agents (e.g., specifically binding antibodies or antigen binding regions, or soluble TWEAK receptor antagonists, ADAM distintegrin domain to antagonize the binding of integrin to its ligands, specifically binding anti-eph receptor and/or anti-ephrin antibodies or antigen binding regions, and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto).

Other anti-angiogenic/anti-tumor agents that can be used in conjunction with the compounds of the present invention include: SD-7784 (Pfizer, USA); cilengitide. (Merck KGaA, Germany, EPO 770622); pegaptanib octasodium, (Gilead Sciences, USA); Alphastatin, (BioActa, UK); M-PGA, (Celgene, USA); ilomastat, (Arriva, USA,); emaxanib, (Pfizer, USA,); vatalanib, (Novartis, Switzerland); 2-methoxyestradiol, (EntreMed, USA); TLC ELL-12, (Elan, Ireland); anecortave acetate, (Alcon, USA); alpha-D148 Mab, (Amgen, USA); CEP-7055, (Cephalon, USA); anti-Vn Mab, (Crucell, Netherlands) DAC: antiangiogenic, (ConjuChem, Canada); Angiocidin, (InKine Pharmaceutical, USA); KM-2550, (Kyowa Hakko, Japan); SU-0879, (Pfizer, USA); CGP-79787, (Novartis, Switzerland); the ARGENT technology of Ariad, USA; YIGSR-Stealth, (Johnson & Johnson, USA); fibrinogen-E fragment, (BioActa, UK); the angiogenesis inhibitors of Trigen, UK; TBC-1635, (Encysive Pharmaceuticals, USA); SC-236, (Pfizer, USA); ABT-567, (Abbott, USA); Metastatin, (EntreMed, USA); angiogenesis inhibitor, (Tripep, Sweden); maspin, (Sosei, Japan); 2-methoxyestradiol, (Oncology Sciences Corporation, USA); ER-68203-00, (WVAX, USA); Benefin, (Lane Labs, USA); Tz-93, (Tsumura, Japan); TAN-1120, (Takeda, Japan); FR-111142, (Fujisawa, Japan); platelet factor 4, (RepliGen, USA); vascular endothelial growth factor antagonist, (Borean, Denmark); bevacizumab (pINN), (Genentech, USA); XL 784, (Exelixis, USA); XL 647, (Exelixis, USA); MAb, alpha5beta3 integrin, second generation, (Applied Molecular Evolution, USA and MedImmune, USA); gene therapy, retinopathy, (Oxford BioMedica, UK); enzastaurin hydrochloride (USAN), (Lilly, USA); CEP 7055, (Cephalon, USA and Sanofi-Synthelabo, France); BC 1, (Genoa Institute of Cancer Research, Italy); angiogenesis inhibitor, (Alchemia, Australia); VEGF antagonist, (Regeneron, USA); rBPI 21 and BPI-derived antiangiogenic, (XOMA, USA); PI 88, (Progen, Australia); cilengitide (pINN), (Merck KGaA, German; Munich Technical University, Germany, Scripps Clinic and Research Foundation, USA); cetuximab (INN), (Aventis, France); AVE 8062, (Ajinomoto, Japan); AS 1404, (Cancer Research Laboratory, New Zealand); SG 292, (Telios, USA); Endostatin, (Boston Childrens Hospital, USA); ATN 161, (Attenuon, USA); ANGIOSTATIN, (Boston Childrens Hospital, USA); 2-methoxyestradiol, (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (p1NN), (GlaxoSmithKline, UK); E 7820, (Eisai, Japan); CYC 381, (Harvard University, USA); AE 941, (Aeterna, Canada); vaccine, angiogenesis, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol, (EntreMed, USA); anginex, (Maastricht University, Netherlands, and Minnesota University, USA); ABT 510, (Abbott, USA); AAL 993, (Novartis, Switzerland); VEGI, (ProteomTech, USA); tumor necrosis factor-alpha inhibitors, (National Institute on Aging, USA); SU 11248, (Pfizer, USA and SUGEN USA); ABT 518, (Abbott, USA); YH16, (Yantai Rongchang, China); S-3APG, (Boston Childrens Hospital, USA and EntreMed, USA); MAb, KDR, (ImClone Systems, USA); MAb, alpha5 betal, (Protein Design, USA); KDR kinase inhibitor, (Celltech Group, UK, and Johnson & Johnson, USA); GFB 116, (South Florida University, USA and Yale University, USA); CS 706, (Sankyo, Japan); combretastatin A4 prodrugs, (Arizona State University, USA); chondroitinase AC, (IBEX, Canada); BAY RES 2690, (Bayer, Germany); AGM 1470, (Harvard University, USA, Takeda, Japan, and TAP, USA); AG 13925, (Agouron, USA); Tetrathiomolybdate, (University of Michigan, USA); GCS 100, (Wayne State University, USA) CV 247, (Ivy Medical, UK); CKD 732, (Chong Kun Dang, South Korea); MAb, vascular endothelium growth factor, (Xenova, UK); irsogladine (INN), (Nippon Shinyaku, Japan); RG 13577, (Aventis, France); WX 360, (Wilex, Germany); squalamine (pIN), (Genaera, USA); RPI 4610, (Sima, USA); heparanase inhibitors, (InSight, Israel); KL 3106, (Kolon, South Korea); Honokiol, (Emory University, USA); ZK CDK, (Schering AG, Germany); ZK Angio, (Schering AG, Germany); ZK 229561, (Novartis, Switzerland, and Schering AG, Germany); XMP 300, (XOMA, USA); VGA 1102, (Taisho, Japan); VEGF receptor modulators, (Pharmacopeia, USA); VE-cadherin-2 antagonists, (ImClone Systems, USA); Vasostatin, (National Institutes of Health, USA);vaccine, Flk-1, (ImClone Systems, USA); TZ 93, (Tsumura, Japan); TumStatin, (Beth Israel Hospital, USA); truncated soluble FLT 1 (vascular endothelial growth factor receptor 1), (Merck & Co, USA); Tie-2 ligands, (Regeneron, USA); and, thrombospondin 1 inhibitor, (Allegheny Health, Education and Research Foundation, USA).

It is contemplated and therefore within the scope of the invention that the compounds of the present invention can be modified to target specific receptors or cancer cells or can be modified so that they can survive various in vivo environments. As examples, when X is a carboxylate functionality, X can be modified so that it is combined with dendrimers or other cyclic sugars to form carboxylate dendrimers or other sugars. It may be combined with steroids such as estrogen to form carboxylate steroids like carboxylate estrogen. X or other carboxylate functionalities on these compounds may be modified so that they contain folic acid. Those of skill in the art will recognize that there are other modifications that can be made to the compounds of the present invention so that they can target specific receptors, cells or provide stability to the compounds. It is contemplated that the compounds of the present invention can have modifications made that are covalent modifications, ionic modifications, modified so that they chelate to other compounds, or other undergo some other type of interaction that allows the compounds of the present invention to suit their use (such as hydrophobic or Van der Waals type interactions).

In a further variation, the compounds of the present invention can be used against solid tumors, cell lines, and cell line tissue that demonstrate upregulated nucleotide excision repair and other upregulated resistance mechanisms.

The PAs of the present invention can be combined with MSNs that are synthesized using cetyltrimethylammonium bromide reacted with tetraethylorthosilicate.

In one variation, the pharmaceutical composition and methods using the composition may contain pharmaceutically acceptable salts, solvates, and prodrugs thereof, and may contain diluents, excipients, carriers, or other substances necessary to increase the bioavailability or extend the lifetime of the compounds/composition of the present invention.

Subjects that may be treated by the compounds and compositions of the present invention include, but are not limited to, horses, cows, sheep, pigs, mice, dogs, cats, primates such as chimpanzees, gorillas, rhesus monkeys, and, humans In an embodiment, a subject is a human in need of cancer treatment.

The pharmaceutical compositions containing the compounds and MSNs of the invention may be in a form suitable for oral use, for example, as tablets, troches, lozenges, aqueous, or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques to form osmotic therapeutic tablets for controlled release.

Formulations for oral use may also be presented as hard gelatin capsules where the active ingredient is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil.

Aqueous suspensions may contain the compounds in the pharmaceutical composition in an admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycethanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and/or flavoring and/or coloring agents.

The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, sterile water for injection (SWFI), Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

In one variation, the formulations of the present invention suitable for parenteral administration may comprise sterile aqueous and non-aqueous injection solutions of the active compound(s), which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s)/composition, or a salt thereof, in a unit dosage form in a sealed container. The compound/composition or salts thereof is provided in the form of a lyophilizate which is capable of being reconstituted with a suitable pharmaceutically acceptable carrier to form a liquid composition suitable for injection thereof into a subject. The unit dosage form may in one variation comprise from about 10 mg to about 10 grams of the compound/composition or salt thereof. When the compound/composition or salt thereof is substantially water-insoluble, a sufficient amount of emulsifying agent which is physiologically acceptable may be employed in sufficient quantity to emulsify the compound or salt in an aqueous carrier. One such useful emulsifying agent that may be used is phosphatidyl choline.

All references cited herein are incorporated herein by reference in their entireties.

### EXAMPLES

### Materials and Methods

### Reagents and Solvents

Cetyltrimethylammonium bromide (CTAB), 3-(aminopropyl)triethoxysilane (APTES), tetraethylorthosilicate (TEOS), and succinic anhydride were purchased from ACROS. 2-[Methoxy(polyethyleneoxy)-propyl]trimethoxysilanes (mPEG-silane; MW=600 and 1,200) were purchased from Gelest (Morrisville, Pa.). mPEG-silanes with MW=5000 and 20,000 were purchased from Laysan Bio (Arab, Ala.). 1,2-Dipalmitoyl-sn-glycero-3-phosphocholine (DPPC), 1,2-dipalmitoyl-sn-glycero-3-phosphoethanolamine-N-[methoxy(polyethyleneglycol)-2000] (ammonium salt) (DSPE-mPEG) and 1,2-dioleoyl-3-trimethylammoniumpropane (chloride salt) (DOTAP) were purchased from Avanti Polar Lipids (Alabaster, Ala.). 1,2-Distearoyl-sn-glycero-3-phosphoethanolamine (DSPE)-conjugated and fluorescein-labeled polyethyleneglycol (DSPE-PEG5k-FITC) was purchased from Nanocs (New York, N.Y.). Compound P1A1 was synthesized and characterized according to previous work done in the inventors lab (Z. Ma, et al., Bierbach, J. Med. Chem. 2008, 51, 7574-7580). All other reagents and solvents were purchased from common vendors and used without further purification, unless stated otherwise.

Synthesis of 4-oxo-4-((3-(triethoxysilyl)propyl)amino)butanoic acid

The “COOH-silane” was synthesized according to a reported procedure with modifications (J. Aburto, et al., Micropor. Mesopor. Mater. 2005, 83, 193-200). A mixture of 440 mg (4.4 mmol) of succinic anhydride and 1.0 mL (4.4 mmol) of APTES in 8.0 mL of dry THF was stirred for 3 h at room temperature. The product was used without further purification after analyzing a sample for quantitative conversion by 1H NMR spectroscopy. 1H NMR (300 MHz, CDCl3) δ6.27 (s, 1 H, NH), 3.83 (q, J=7.0 Hz, 6 H, CH2O), 3.27 (q, 2 H, CH2NH), 2.72-2.65 (m, 2 H, CO(CH2)2CO), 2.54-2.48 (m, 2 H, CO(CH2)2CO), 1.70-1.58 (m, 2 H, CH2), 1.23 (t, J=7.0 Hz, 9 H, CH3), 0.64 (t, 2 H, SiCH2).

### Synthesis of MSN

The MSN materials were synthesized according to published procedures with modifications (Q. Cai, et al., Chem. Mater. 2001, 13, 258-263; S. Gin, et al., Angew. Chem. Int. Ed. Engl. 2005, 44, 5038-5044). To 0.156 g of CTAB dissolved in 75 mL of water in a 250 mL round bottom flask were added 0.54 mL of 2M NaOH, n-decane, and DMF and the mixture was stirred for 1 h at 80° C. To the vigorously stirred mixture were added 0.78 mL of TEOS at a rate of 0.07 mL/min using a syringe pump, and stirring was continued at 500 rpm for another 2 h. The products (CTAB@MSN) were collected by centrifugation (19,000 rpm, 10 min), washed 2× with water and ethanol, and dried in a vacuum for 12 h.

### Synthesis of MSN-COOH and P1A1@MSN-COOH

The template in CTAB@MSN was removed by heating 300 mg batches of the material 3× for 12 h at reflux in conc. HCl/ethanol (0.5 mL/50 mL). The material (MSN) was collected by centrifugation (19,000 rpm, 10 min), washed 2× with ethanol, and dried in a vacuum overnight. To 100 mg of MSN, suspended with sonication in 10 mL of toluene, was then added 1 mL of the freshly prepared COOH-silane in THF (0.5 mM), and the mixture and stirred and refluxed overnight. MSN-COOH was collected by centrifugation (19,000 rpm, 10 min), washed 2× with ethanol, and dried in a vacuum overnight. To generate P1A1@MSN-COOH with a specific payload content, MSN-COOH (1 mg/mL) was dispersed in 0.1-5.0 mM P1A1 in PBS, and the mixture was incubated with gentle agitation for 24 h at room temperature. Drug-loaded nanoparticles were collected by centrifugation (13,400 rpm, 5 min), washed 2× with water and allowed to dry in air overnight.

### Synthesis of MSN-COOH-PEG and P1A1@MSN-COOH-PEG

CTAB@MSN (250 mg) was stirred in 20 mL of toluene until thoroughly dispersed (30 min). 50 mg of mPEG-silane was added and the mixture was stirred for 30 min at room temperature and then heated at reflux for 12 h. The resulting CTAB@MSN-PEG materials were collected by centrifugation (19,000 rpm, 10 min). CTAB was removed to generate MSN-PEG, which was washed, dried, and treated with COOH-silane to produce MSN-COOH-PEG using the same procedure as described for MSN-COOH. P1A1@MSN-COOH-PEG was then generated as described for P1A1@MSN-COOH.

### Lipid Film Preparation and Synthesis of MSN-COOH-LIP and P1A1@MSN-COOH-LIP

Stock solutions of DPPC, DOTAP, and DSPE-mPEG were prepared in CDCl₃ at concentrations of 10 mg/mL. The desired ratios of lipids for LIP10 (75:10:5 wt %) and LIP20 (85:20:5 wt %) were mixed in a 25 mL round bottom flask and sonicated for 5 min to form a clear solution. Rotary evaporation of solvent was used to generate lipid thin films, which were dried at room temperature for 6 h and stored at 4° C. until use. For particles used in confocal microscopy studies, 50% of the DSPE-mPEG was replaced with DSPE-PEG-FITC. Batches of MSN-COOH-LIP and P1A1@MSN-COOH-LIP were prepared according to a published procedure with minor changes as follows (H. Meng, et al., ACS Nano 2015, 9, 3540-3557): a suspension of 2.0 mg of MSN-COOH in 1 mL of PBS solution was transferred to a 25 mL round bottom flask containing 5 mg of dry lipid film. After incubation in a water bath at 50° C. for 30 min (above the phase transition temperature of DPPC, 41° C.), the sample was sonicated for 20 min in short 1 min on/1 min off intervals using a thermostatted Fisher Scientific CPX Ultrasonic bath. The resulting MSN-COOH-LIP particles were collected by centrifugation (13,400 rpm, 5 min) and washed with PBS and centrifuged twice to remove excess lipid. P1A1@MSN-COOH-LIP was generated and isolated in the same manner from P1A1@MSN-COOH. Both materials were stored in water or PBS solution at 4° C.

### Characterizations of Materials

Transmission Electron Microscopy (TEM) images in bright-field mode were captured on an FEI Tecnai 12 BioTWIN transmission electron microscope at an accelerating voltage of 80 kV. MSNLP size distribution was determined from TEM images for n>200 particles using ImageJ (version 1.51f, National Institutes of Health, Bethesda, Md., 2016). Scanning Transmission Electron Microscopy (STEM) was performed using a probe-corrected FEI Titan at 200 kV acceleration voltage. The probe convergence angle was set to 19.6 mrad and the beam current at ˜100 pA to mitigate issues of beam-induced sample damage. High-angle annular dark field (HAADF) imaging was used to study the shape and physical morphology of the specimens, and energy dispersive X-ray spectroscopy (EDS) was used to study the chemical composition of the sample. The EDS detector consisted of four Si-Li detectors with a total collection angle of 0.9 sr, which is significantly larger than traditional X-ray detectors, thereby reducing the acquisition times for typical EDS maps to a few minutes. Samples for TEM and STEM analysis were dispersed in water and spotted onto formvar/carbon-coated copper grids (Ted Pella Inc.) and allowed to air-dry. For lipid-coated MSN samples, uranyl acetate (UA) was used as a negative stain to enhance image contrast. FTIR spectra were recorded on a Perkin Elmer Spectrum 100 spectrometer equipped with an ATR accessory. TGA traces were recorded on a TA SDT Q600 thermogravimetric analyzer in air at a heating rate of 10° C./min Samples were lyophilized prior to TGA measurements. The ζ-potential and size distribution of the nanoparticles were determined on a Malvern Zetasizer Nano ZS 90 analyzer. Small-angle X-ray scattering data were acquired on a Bruker D8 Discover diffractometer configured with CuKα radiation, parallel beam optics (Göbel mirror), and LynxEye detector with a step size of 0.01° and a speed of 1 s/step. Nitrogen sorption isotherms were recorded using a Micromeritics Tristar II surface area and porosimeter analyzer. Samples were thoroughly degassed at 200° C. for at least 6 h. The surface area and pore size distribution were calculated using Brunauer-Emmett-Teller (BET) and non-local density functional theory (NLDFT) methods, respectively. Pore volumes were calculated at a relative pressure, P/P0, of 0.9. P1A1 was quantified from UV-vis absorbances at λmax=413 nm recorded on a Synergy BioTek H1 plate reader and appropriate calibration curves.

### In Vitro Drug Release

Drug-loaded nanoparticles (1 mg) were suspended in 1 mL of PBS (pH 7.6) or sodium acetate buffer (pH 4.6, [chloride]=5 mM) at 37° C. in a Labnet Accublock digital dry bath. At each time point (0.25, 2, 4, 6, 12, 24, 48, 72 h) 100 μL of the colloidal dispersion was removed and immediately centrifuged at 13,400 rpm for 5 min. The supernatant was analyzed for released P1A1 content as described above. Measurements were performed in quadruplicate in a 96-well plate format.

### Cell Culture

BxPC3 (CRL-1687) and PANC-1 (CRL-1469) cells were obtained from and authenticated by American Type Culture Collection (Manassas, Va.).

BxPC3 cells were maintained in complete medium consisting of RPMI-1640 (Lonza, Basel, Switzerland) supplemented with 10% fetal bovine serum (FBS, Sigma Aldrich, St. Louis, Mo.), 100 IU/mL penicillin (Life Technologies, Carlsbad, Calif.) and 100 μg/mL streptomycin (Life Technologies, Carlsbad, Calif.). PANC1 cells were maintained in Dulbecco's Modified Eagle's Medium (DMEM, Lonza, Basel, Switzerland) supplemented with 10% FBS, 100 IU/mL penicillin and 100 g/mL streptomycin. All cells were used within six months of resuscitation.

### Cell Proliferation Assay

BxPC3 or PANC-1 (5×104 cells) were seeded in 96-well tissue culture plates (BD Falcon, San Jose, Calif.) and allowed to attach overnight. Cells were treated as indicated for 72 h. Medium was aspirated and replaced with appropriate complete medium containing thiazolyl blue tetrazolium bromide, MTT (0.5 mg/mL) (Sigma Aldrich, St. Louis, Mo.). Plates were incubated at 37° C. in 5% CO2 for 45-75 minutes and then the medium was replaced with dimethyl sulfoxide (Fisher Science, Fairlawn, N.J.). Wells were mixed using a micropipette and absorbance was read at 560 nm and corrected using a reference wavelength of 650 nm using a Molecular Devices (Sunnyvale, Calif.) Emax Precision Microplate Reader.

### Flow Cytometry

BxPC3 cells were treated with P1A1@MSN_{LP}-COOH-LIP₁₀, P1A1, or MSNLP-COOH-LIP₁₀ for 48 h at the indicated concentrations. Cells were gently trypsinized, washed in PBS, fixed in ice-cold EtOH:PBS (1:1) at equivalent cell concentrations, and stored at −20° C. Cells were washed with PBS, stained using FxCycle PI/RNase staining solution (Molecular Probes, Eugene, Oreg.) and assessed by flow cytometry using an Accuri C6 (BD Biosciences, San Jose, Calif.). Data was analyzed using Modfit software v3.3.

### Confocal Microscopy

BxPC3 cells were seeded onto chamber slides (Nunc, Rochester, N.Y.) and allowed to attach for 48 h. Cells were then treated with 5 μM P1A1, P1A1@MSN_{LP}-COOH-LIP₁₀ at an equivalent concentration of drug, or vehicle. The medium was aspirated and cells were co-stained by incubating with 75 nM Lysotracker Red in complete medium for 1 h. Cells were washed 3× with PBS and fixed in 4% formaldehyde/PBS for 15 min. After another three PBS washes, cover slips were mounted onto 4-well chamber slides using vectashield HardSet mounting medium (Vector Labs, Burlingame, Calif.) and stored at 4° C. until ready to use. Images were collected using a Zeiss LSM 710 confocal microscope (Carl Zeiss MicroImaging, Thornwood, N.Y.) using a 63× (PLAN Oil APO, 1.4 NA) objective lens. All image channels were acquired in a sequential mode to minimize excitation cross talk and emission bleed-through. A 405 nm laser line (for P1A1)) was utilized with an emission range of 410-475 nm, a 488 nm laser line (for FITC) with an emission range of 494-542 nm, and a 561 nm laser line (for LysoTracker Red) with an emission range of 572-690 nm. For comparative fluorescence intensity analysis, great care was taken to equalize excitation power, pinhole settings, PMT gain, and offset values across and within imaging sessions for each respective channel. For all images, the pinhole value was kept at or below 1.2 airy units, and images were acquired with 233 line averaging at 1832×1832 pixels. Zen software was used for image acquisition. Where necessary, post-acquisition contrast adjustments were applied to the entire image, and processing was identical in all fluorescence channels. Image panels were assembled and annotated in Photoshop CS2 without any additional manipulation.

### Results

### Preparation and Characterization of a Large-Pore MSN Host Material (MSNLP)

To accommodate P1A1 (longest dimension ˜1.6 nm, estimated from its X-ray crystal structure) in addition to grafted auxiliary functional groups on the interior surface, an initial pore inner diameter of greater than 6 nm was desired (Z. Ma, et al., J. Med. Chem. 2008, 51, 7574-7580). Using sol-gel chemistry and a unique combination of swelling agent and co-solvent MSNLP (where ‘LP’ stands for large-pore) was generated, and was found to be morphologically distinct from classical MCM-41 (C. T. Kresge, et al., Nature 1992, 359, 710-712).

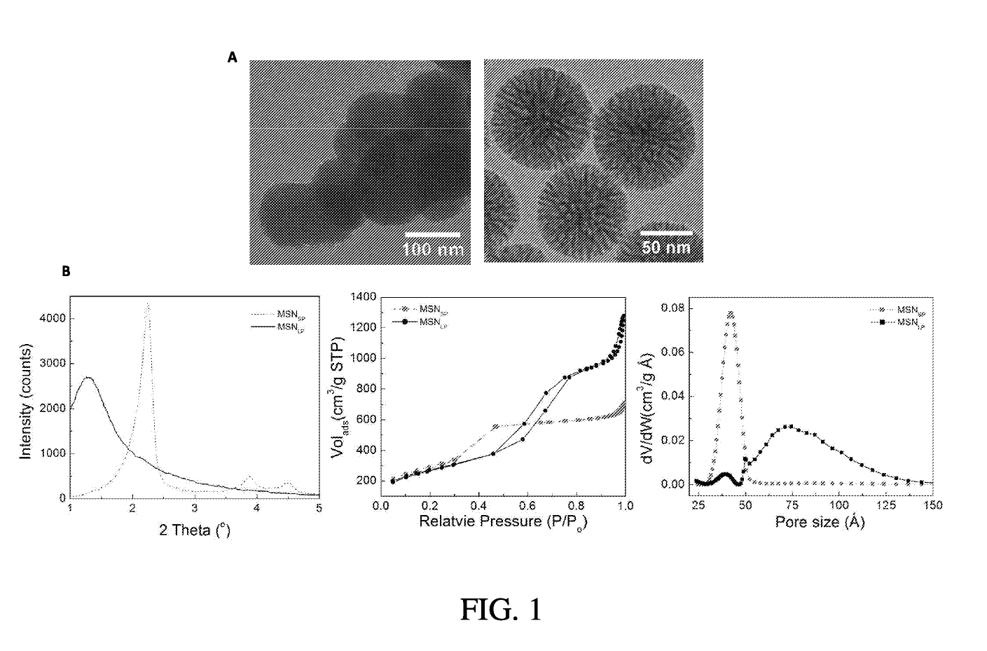

The procedure involved the use of micelle-forming CTAB soft template and n-decane as a swelling agent in dilute NaOH solution in the presence of DMF as an aprotic, high-boiling co-solvent. DMF promoted formation of significantly larger pore diameters than commonly used ethanol (K.-C. Kao and C.-Y. Mou, Micropor. Mesopor. Mat. 2013, 169, 7-15). These particles, unlike MSNSP (where ‘SP’ stands for small-pore) appear as ideal spherical shapes in transmission electron microscopy (TEM) images (see FIG. 1A). Using a 1:1 (v/v) mixture of n-decane and DMF, a material was generated with interconnected, disordered mesopores with an average diameter of ˜7 nm, based on nitrogen physisorption measurements and small-angle X-ray scattering (SAXS) data (see FIG. 1B). Unlike other large-pore MSN materials, which often show bimodal pore distributions (D. Niu, et al., Adv. Mater. 2014, 26, 4947-4953), MSNLP are characterized by a unimodal distribution of mesopores in the range of about 5-15 nm. MSNLP also maintains a large surface area (957 m2/g) and pore volume (1.44 cm3/g). By contrast, MSNSP showed the characteristic features (C.-Y. Chen, et al., Micropor. Mat. 1993, 2, 17-26) of an ordered two-dimensional honeycomb hexagonal lattice and a narrow pore size distribution with an average pore diameter of 4 nm (see FIG. 1B). The large-pore nanoparticles (MSNLP) generated in this manner that measured 105±25 nm in diameter were used for further modification and drug loading.

Drug-Loaded Carboxylate-Modified Materials (P1A1@MSN-COOH)

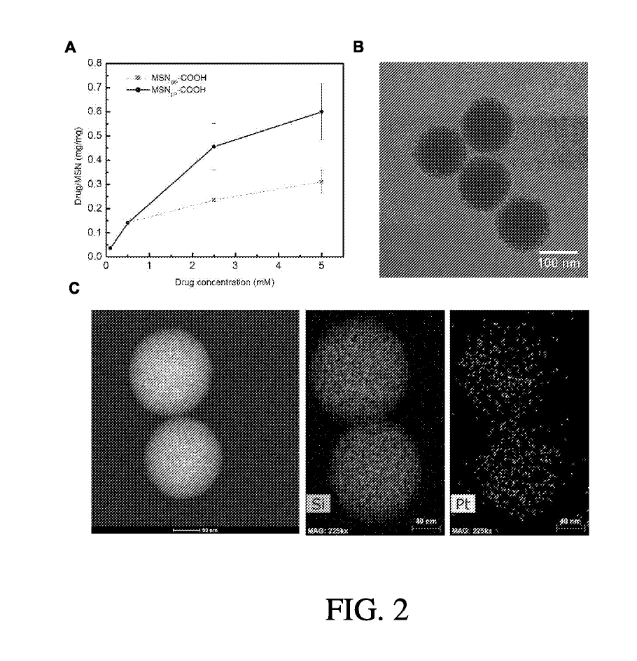

In one embodiment, the effect of pore size on loading efficiency of P1A1 was studied. Precursors MSNSP-COOH and MSNLP-COOH were generated from the corresponding MSN materials by grafting carboxylate functionalities onto the pore surfaces using 4-oxo-4-((3-(triethoxysilyl)propyl)amino)butanoic acid (“COOH-silane”, synthesized from 3-aminopropyltriethoxysilane/APTES, and succinic anhydride). When these materials, without further modification, were incubated in buffered aqueous solutions of varying concentrations of P1A1, uptake of payload by MSNLP-COOH was twice as efficient as that observed for MSNSP-COOH (see FIG. 2A). The materials recovered from the most concentrated solutions of P1A1 used in this study contained approximately 40 wt. % P1A1 payload. Thus, in an embodiment, payloads of about 10 wt. % P1A1 to 40 wt. % P1A1 are contemplated and within the scope of the invention. Alternatively, amounts of about 20 wt. %, or of about 30 wt. % of P1A1 are contemplated. Representative TEM and scanning transmission electron microscopy (STEM) images of P1A1@MSN_{LP}-COOH are shown in FIGS. 2B and C. A sample generated by allowing MSNLP-COOH to soak in 0.5 mM P1A1 was further analyzed for elemental composition by energy-dispersive X-ray spectroscopy (EDS) (see FIG. 2C). On the basis of the EDS elemental mapping data, a Pt content of 3.4 wt. % was estimated to be present in this sample of P1A1@MSN_{LP}-COOH, which corresponds to 12 wt. % of P1A1. This result is in excellent agreement with the value determined spectrophotometrically for the hybrid payload in uptake experiments (see FIG. 2A). Although the present invention contemplates the mix of PAs with MSNLP particles, the drug-containing nanoparticles generated tend in some cases to agglomerate in water and in buffers of biologically relevant pH and ionic strength (studied using dynamic light scattering, DLS). Therefore, in an embodiment, the present invention relates to PA and MSN_{LP}s in combination with additional modifications that improve their biocompatibility (Y. Zhu, et al., Micropor. Mesopor. Mat. 2011, 141, 199-206; L.-S. Wang, et al, ACS Nano 2010, 4, 4371-4379).

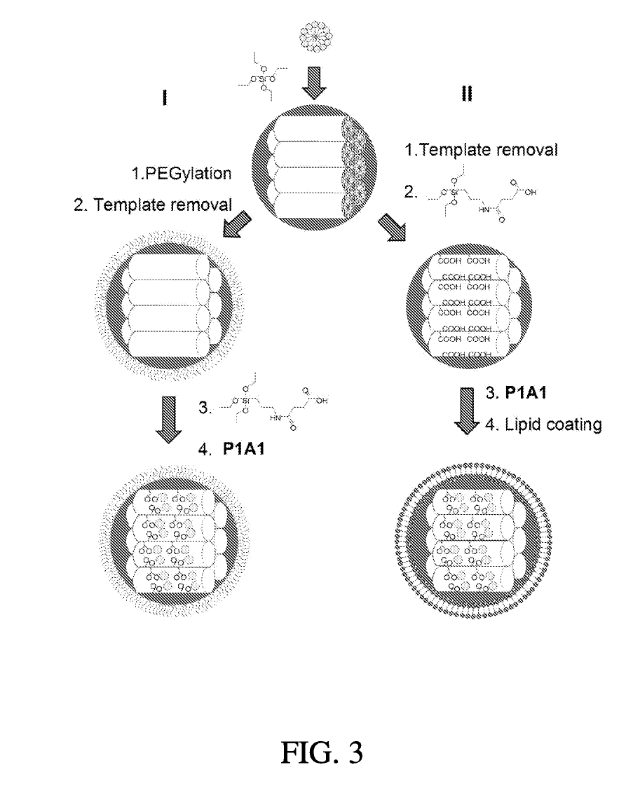

With the desired carboxylate-functionalized MSN_{LP} (MSN_{LP}-COOH) in hand, two strategies of improving the colloidal stability of the nanoparticles were explored: the first method involved covalent PEGylation (MSN_{LP}-COOH-PEG) (see FIG. 3, synthetic route I), and the second utilized coating of the material with a lipid bilayer (MSN_{LP}-COOH-LIP) (see FIG. 3, synthetic route II).

### PEGylated, Carboxylate-Modified Materials (P1A1@MSN-COOH-PEG)

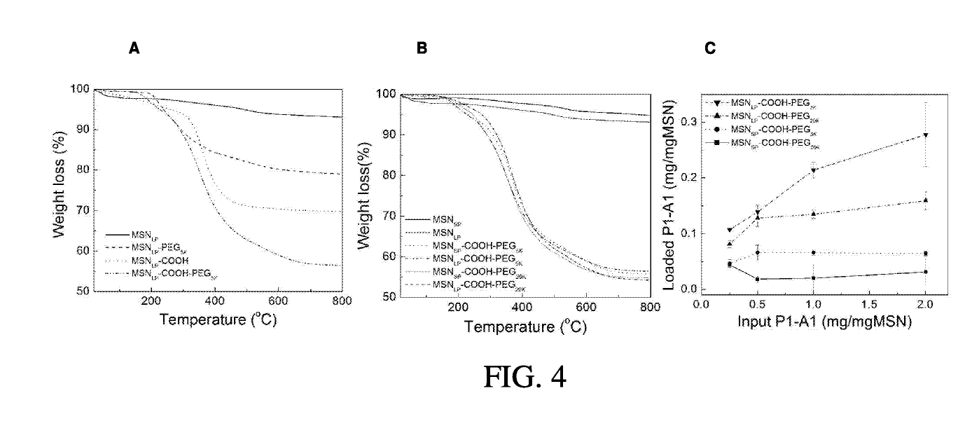

PEGylation increases the aqueous dispersibility and prolongs circulation times of nanoparticles in blood, while reducing their removal from circulation by the mononuclear phagocyte system (S. G. Antimisiaris, P. Kallinteri, D. G. Fatouros in Pharmaceutical Manufacturing Handbook: Production and Processes (Ed.: S. C. Gad), John Wiley & Sons, Inc., Hoboken, N.J., USA 2008, pp. 443-533). Thus, the first strategy pursued was covalent PEGylation to generate MSN-COOH-PEG nanoparticles from MSNLP (for comparison, analogous materials were also generated from MSNSP). This was achieved by sequential grafting (FIG. 3, synthetic route I) of (i) 2-[methoxy(polyethyleneoxy)propyl] trimethoxysilane (“mPEG-silane”, molecular weight 5,000 or 20,000) and (ii) COOH-silane. To minimize physical blocking of pores by PEG and depletion of reactive silanol (Si—OH) groups on the interior pore walls required for subsequent installation of carboxylic acid groups, PEGylation was performed prior to acid-extraction of CTAB from the mesochannels. Using optimized relative amounts of mPEG5k/20k-silane and COOH-silane, 4 materials were generated from MSNSP and MSNLP and characterized by thermogravimetric analysis (TGA) (see FIG. 4), as well as nitrogen physisorption measurements, dynamic light scattering analysis (DLS), and FT-IR spectroscopy. The TGA data confirm that the procedure resulted in materials of well-defined composition, based on the relative ratios of the grafted components (FIGS. 4A and B).

In incubations with P1A1, the loading efficiency of the MSNLP materials was superior to that of the corresponding MSNSP materials, and samples modified with PEG5k groups were able to bind significantly more drug than those modified with PEG20k groups (see FIG. 4C). Overall, the highest payload achieved in these materials was observed for MSNLP-COOH- PEG5k, reaching approximately 20 wt. % at the highest concentration of P1A1 tested, which is half the amount of drug associated with non-PEGylated MSNLP-COOH. This finding and the trends observed in FIG. 4C are in complete agreement with the results of nitrogen physisorption measurements on the four materials: dual modification results in reduced BET surface areas and pore dimensions relative to unmodified MSN, and the effects are more pronounced for MSNSP than for MSNLP. Furthermore, the surface area found in MSNLP containing PEG20k was reduced by approximately 50% compared to that in the material modified with PEG5k, consistent with the observed differences in loading efficiency. While both large-pore materials maintain pore diameters of 5-6 nm, the small-pore materials show a loss of pore volume, most likely due to pore blockage by the grafted functionalities, and the data did not lend itself for calculating meaningful pore diameters.

PEGylation results in nanoparticles with improved hydrodynamic parameters (less aggregation/sedimentation) compared to the MSNSP/LP-COOH materials. In phosphate-buffered saline (PBS), the MSNLP- COOH-PEG5k and MSNLP-COOH-PEG20k preparations show diameters in the range 250-350 nm, which is significantly larger than the dimensions determined for MSNLP-COOH by TEM and STEM. This behavior suggests altered hydrodynamic properties due to changes in microviscosity or some level of particle clustering. (The use of PEG600 and PEG1.2k resulted in even larger aggregates/clusters of >600 nm). However, PEGylation was considered to be less ideal than other methods for improving the biocompatibility of the MSNLP-COOH particles because it failed to promote long-term colloidal stability and limited to a small extent the drug loading capacity of the material.

Lipid-Coated Materials (P1A1@MSN-COOH-LIP): Colloidal Stability and Drug Retention/Release Characteristics

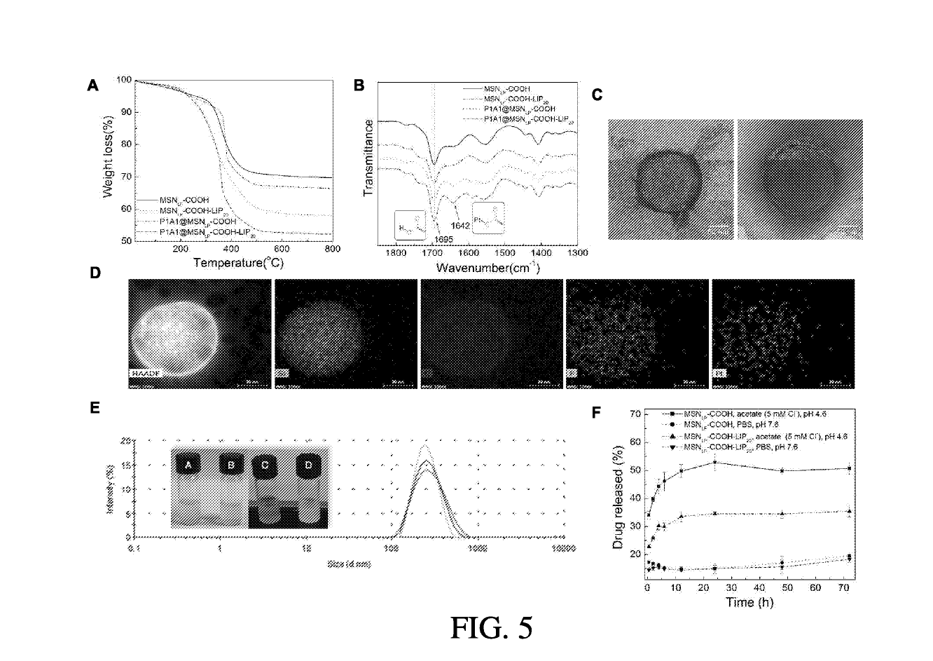

An alternative strategy to generate stable dispersions of nanomaterials in high-ionic-strength media is to introduce PEGylated lipids as surface coatings, a method developed by the inventors to encapsulate and increase the colloidal stability of anionic nanoparticles including viruses (R. Singh, et al., FASEB J. 2008, 22, 3389-3402) and drug-loaded carbon nanotubes (C. D. Fahrenholtz, et al., J. Inorg. Biochem. 2016). Others investigated this technique for encapsulation of MSNs (C. E. Ashley, et al., Nat. Mater. 2011, 10, 389-397 ; J. Liu, et al., J. Am. Chem. Soc. 2009, 131, 7567-7569). This form of MSN has recently shown improved biocompatibility and safety as a delivery vehicle for the prodrug irinotecan compared to liposomal nanoencapsulation alone (X. Liu, et al., ACS Nano 2016, 10, 2702-2715). The procedure applied here for P1A1 involved soaking MSN_{LP}-COOH material with payload and coating of the drug-loaded particles with a lipid bilayer using conventional lipid thin film hydration (Z. A. Mohammadi, et al., Curr. Drug Deliv. 2015) followed by removal of excess lipid and unencapsulated drug (see FIG. 3, synthetic route II). After prescreening a small library of more than 30 lipid compositions to identify formulations that minimized nanoparticle aggregation or sedimentation in PBS, two promising formulations were selected to generate mixtures of zwitterionic and cationic lipids. 1,2-dipalmitoyl-sn-glycero-3-phosphocholine (DPPC), 1,2-dioleoyl-3-trimethylammoniumpropane (DOTAP), and 1,2-dipalmitoyl-sn-glycero-3-phosphoethanolamine-N-[-[-methoxyethyleneglycol)-2000 (DSPE-mPEG2k): P1A1@MSN_{LP}-COOH-LIP₁₀ and P1A1@MSN_{LP}-COOH-LIP₂₀ were used, where P1A1@MSN_{LP}-COOH-LIP₂₀ and the corresponding drug-free material, MSNLP-COOH-LIP₂₀, were fully characterized by TGA, FT-IR, TEM, STEM-EDS, and DLS. The former two methods confirmed the compositions of the multi-component systems (see FIG. 5A and B). A comparison of FT-IR spectra recorded for lipid-encapsulated and unencapsulated materials before and after loading with P1A1 show distinct differences in the C═O stretching frequencies (see FIG. 5B). All samples show an intense band at 1695 cm-1 that can be assigned to v(C═O) of the free carboxylic acid (with contributions from amide carbonyl) groups, while materials containing P1A1 show an additional feature at 1642 cm-1, which is typically observed for monodentate carboxylato ligands in platinum(II) complexes (K. Nakamoto, Infrared and Raman Spectra of Inorganic and Coordination Compounds, Part B, John Wiley & Sons, Inc., New Jersey, USA 2009, pp. 62-72). Thus, the FT-IR data suggest that the grafted carboxylate groups are to some extent involved in coordinative bonding with the payload, which is corroborated by the elemental composition obtained from the EDS analysis of the samples (see below).

TEM images captured of the drug-free and drug-modified materials stained with uranyl acetate (UA) show an intact lipid bilayer (FIG. 5C) approximately 6-7 nm thick. Dark-field STEM images and EDS maps captured of P₁A1@MSN_{LP}-COOH-LIP₂₀ confirm the expected morphology and elemental composition of the nanoparticles (FIG. 5D). The latter data suggest that only 20% of the payload exists in the form of intact monochloro complex P1A1, consistent with the majority of the chloro ligands substituted by grafted carboxylate groups and subsequent loss of chloride from the material. This was confirmed by additional STEM images, EDS spectra, and a summary of the elemental composition.

Modification of the MSN material with a lipid bilayer significantly improves the particles' dispersibility in water and PBS solution. P1A1@MSN_{LP}-COOH-LIP₂₀ forms dispersions with average hydrodynamic particle diameters of 220-250 nm and polydispersity indices (PDI) of less than 0.1, characteristic of a monodisperse distribution of particles, which persists virtually unchanged after 60 min in high-ionic-strength buffered media (see FIG. 5E). Unlike MSN_{LP}, MSN_{LP}-COOH, and P1A1@MSN_{LP}-COOH, the P1A1@MSN_{LP}-COOH-LIP₂₀ particles show a positive ζ-potential of 44 mV, consistent with a particle surface coated with cationic lipids. Without being bound by theory, this feature and steric hindrance due to PEG are likely responsible for the enhanced colloidal stability of the dispersions. Samples of MSN_{LP}-COOH-LIP₂₀ and P1A1@MSN_{L}-COOH-LIP₂₀ stored in PBS for several weeks at 4° C. (FIG. 5E, inset) can be easily redispersed with minor sonication. The acridine fluorophore of P1A1 in the latter material shows intense blue fluorescence when irradiated with 365-nm UV light (see inset in FIG. 5E), which was exploited in confocal microscopy studies to track nanoparticle uptake and payload release in pancreatic cancer cells.

Prior to the cell-based assays, the equilibrium release of P1A1 from P1A1@MSN_{LP}-COOH-LIP₂₀(40 wt. % PA content) were studied as well as the corresponding lipid-free material, P1A1@MSN_{LP}-COOH, in relevant buffers mimicking extracellular conditions (PBS, pH 7.6, containing 140 mM chloride) and intracellular conditions in the lysosomes (acetate buffer, pH 4.6, 5 mM NaCl). The two materials were incubated in buffers for 72 h at 37° C., and samples were withdrawn from the suspensions at appropriate time intervals, centrifuged, and the supernatant analyzed spectrophotometrically for release of P1A1. Neither the lipid-free nor the lipid-coated material showed a significant release of drug over time in PBS (see FIG. 5F). Both mixtures show a constant level of P1A1, which was approximately 15% of the payload originally associated with the nanoparticles, during the 3 days of incubation. This observation is consistent with the dissociation of only a small amount of P1A1 adsorbed on the surface of the particles, but not from the interior pores. Thus, leakage over time of P1A1 from the materials in a high-chloride environment and neutral pH can be considered slow and negligible, which supports the design rationale. This is an important finding since similar pH-sensitive carboxylate-modified (small-pore) materials have shown significant release of payload under similar conditions (J. Gu, et al., J. Phys. Chem. Lett. 2010, 1, 3446-3450; S. Ramasamy, et al., RSC Advances 2015, 5, 79616-79623). Most importantly, the fact that no difference in drug release is observed for P1A1@MSN_{LP}-COOH-LIP₂₀ and P1A1@MSN_{LP}-COOH demonstrates that at neutral pH and physiological chloride concentration the lipid bilayer is not required as a diffusive barrier to seal the pores and prevent leakage of drug. Likewise, the above study showed that material does not require capping of the pores with small-molecules or nanoparticles (“gated pores”) (C. Coll, et al., Acc. Chem. Res. 2012, 46, 339-349; J. Pang, et al., J. Colloid Interface Sci. 2012; b) M. R. Zeiderman, et al., ACS Biomat. Sci. Eng. 2016, 2, 1108-1120; J. Zhang, D. Desai and J. M. Rosenholm, Adv. Funct. Mater. 2016, 24, 2352-2360) to prevent premature release of payload.

An entirely different situation was observed when the two materials were dispersed in an acetate buffer of pH 4.6 (supplemented with 5 mM NaCl to mimic the relatively lower level of chloride observed in the cytosol and acidic organelles of cells) (Y. Ishida, et al., J. Gen. Physiol. 2013, 141, 705-720). Both P1A1@MSN_{LP}-COOH and P1A1@MSN_{LP}-COOH-LIP₂₀ show release of approximately 50% and 35% of payload, respectively, with the maximum level reached after 24 h of incubation. Under these equilibrium conditions the lipid bilayer appears to act as a diffusion barrier favoring higher payload retention in the particles. Approximately half of the free drug is generated during the first 20 min of incubation, suggesting rapid release of payload triggered by the acidic pH.

### Activity and Mechanism of Action in Human Cancer Cells

Without being bound by theory, the proposed mechanism of action of P1A1@MSN-COOH-LIP nanoparticles involves (i) endocytosis and accumulation in endosomes, (ii) release of P1A1 in, and escape from, the acidic lysosomes, and (iii) diffusion of payload into the nucleus to form cytotoxic DNA adducts. To test the functionality of the nanocarrier at the cellular level, the effects of the PA-loaded materials in two pancreatic cancer cell lines, PANC-1 and BxPC3 were studied, using a colorimetric cell proliferation (MTT) assay in combination with flow cytometry and confocal fluorescence microscopy. Both cell lines harbor homozygous p53 mutations (American Type Culture Collection (ATCC), Manassas, Va., USA 2013), and the former cell line has previously shown significantly higher sensitivity to PAs than to the clinical drug cisplatin (L. A. Graham, et al., J. Med. Chem. 2012, 55, 7817-7827).

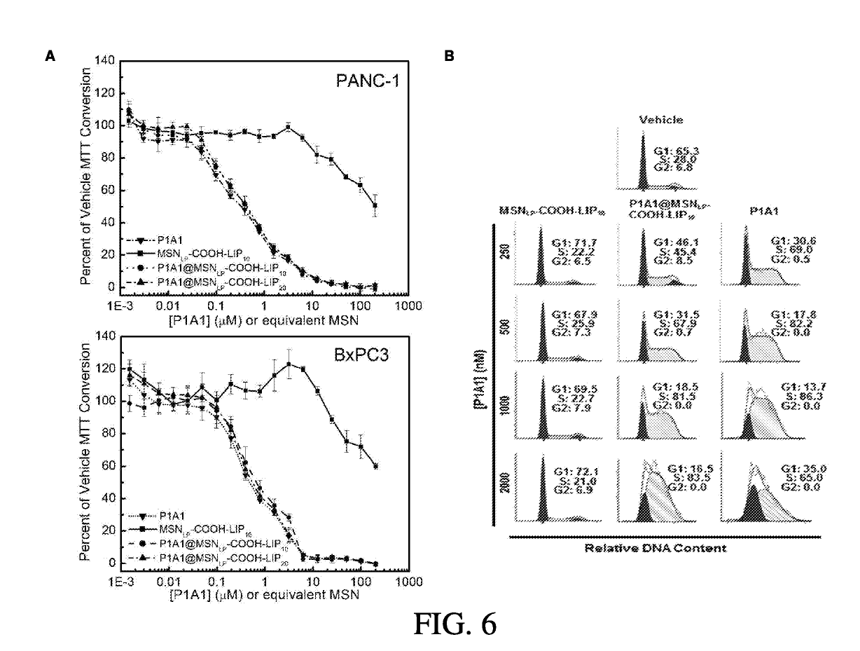

PANC-1 and BxPC3 cells were treated for 72 h with varying concentrations of P1A1, or equivalent P1A1@MSN_{LP}-COOH-LIP₁₀ and P1A1@MSN_{LP}-COOH-LIP₂₀ (40 wt. % P1A1), as well as the drug-free carrier material, MSN_{LP}-COOH-LIP₁₀. In both cell lines, P1A1 and its two nanocarrier formulations reduced cell viability with submicromolar IC50 values in the range 0.3-0.8 μM (see FIG. 6A). This level of potency is consistent with that observed previously for PAs in PANC-1, which proved to be 10-70-fold higher than the cytotoxicity levels achieved with cisplatin (L. A. Graham, et al., J. Med. Chem. 2012, 55, 7817-7827). By contrast, the PA-free carrier MSN_{LP}-COOH-LIP₁₀ was at least two orders of magnitude (IC50>100 μM) less cytotoxic to the cancer cells under the same conditions, confirming that the loss of cell viability observed after treatment with P1A1@MSN nanoparticles was solely caused by the payload. These data, coupled with the extracellular stability of the P1A1 loading (FIG. 5F), suggested that the nanoparticles must have been internalized and the payload released intracellularly.

To gain further insight into the mechanism of cell death of nanocarrier-delivered P1A1, flow cytometry analysis was performed with BxPC3 cells treated with P1A1@MSN_{LP}-COOH-LIP₁₀. Dosing was done with micromolar-to-submicromolar concentrations for 48 h, and the effect on cell cycle progression was compared with that of MSN_{LP}-COOH-LIP₁₀ and free P1A1 (FIG. 6B). Drug-free carrier, even at the highest concentration tested, had no effect on the cell cycle distribution of the treated cells, which was virtually the same as that observed for control (vehicle), with approximately 70% of the cells in G1 phase. By contrast, cells treated with P1A1 or P1A1@MSN_{LP}-COOH-LIP₁₀show a robust S phase arrest (FIG. 6B).

The fraction of cells in S phase increased with concentration of both free and nanoparticle-delivered P1A1 while the population of G2 cells completely disappeared. For P1A1, cells began to show a sub-G1 population at the highest dose, indicative of cellular debris due to DNA fragmentation as a result of cell death. P1A1@MSN_{LP}-COOH-LIP₁₀ and P1A1 achieve a similar maximum level of S phase arrest (>80% of the cell population). These results seem to suggest that free and carrier-delivered P1A1 act by the same molecular mechanism at the target level. Without being bound by theory, PAs cause cell death by forming high levels of monofunctional-intercalative adducts in genomic DNA (X. Qiao, et al., Metallomics 2012, 645-652). These adducts are an intrinsically more severe form of DNA damage than the cross-links formed by DNA, and specialized DNA double-strand repair endonucleases are required for their removal (F. Liu, et al., Chem. Res. Toxicol. 2015, 28, 2170-2178). Cells treated with PAs show cell cycle arrest in late G1/early S phase as the result of this type of DNA damage, one of the hallmarks of PA-induced cancer cell death (C. L. Smyre, et al., ACS Med. Chem. Lett. 2011, 2, 870-874).

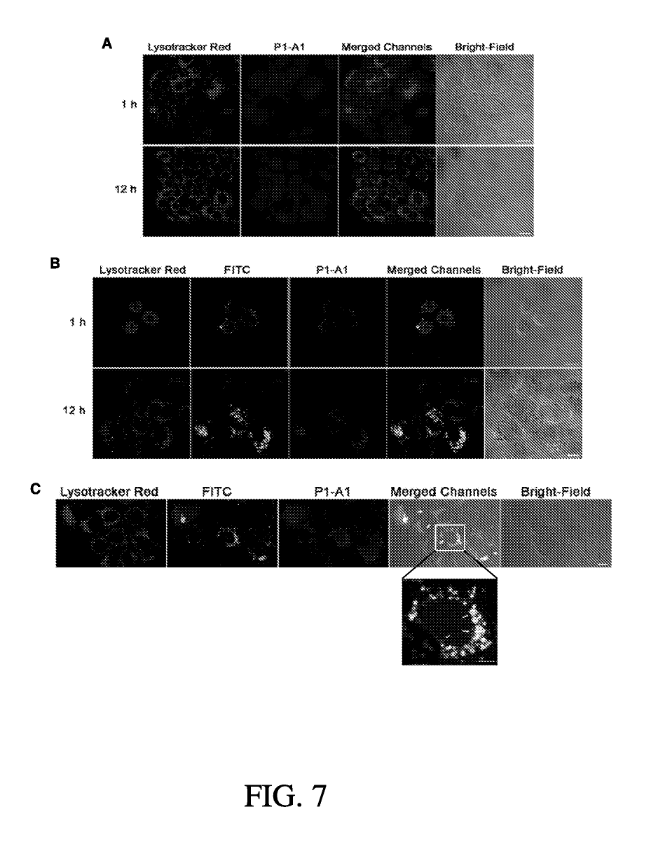

To compare the internalization and subcellular trafficking of P1A1 alone and as payload of nanoparticles, a colocalization study was performed using confocal fluorescence microscopy. This was possible by taking advantage of the intrinsic blue fluorescence of the 9-aminoacridine chromophore in P1A1 and selective staining of the acidic lysosomes with pH-sensitive Lysotracker Red. To detect intracellular P1A1, in particular in the nucleus where the fluorescence of DNA-associated acridine is known to be severely quenched (S. Ding, et al., J. Med. Chem. 2012, 55, 10198-10203), it was necessary to treat cells at relatively high concentrations (5 μM) of the drug, but only for short periods of time to avoid cell death. Images of BxPC3 cells treated with P1A1 for 1 h and 12 h are shown in FIG. 7A. As expected from the inventors' previous studies in lung cancer cells (A. J. Pickard, et al., Chem. Eur. J. 2014, 20, 16174-16187; X. Qiao, et al., Metallomics 2012, 645-652; F. Liu, et al., Chem. Res. Toxicol. 2015, 28, 2170-2178; S. Ding, et al., Angew. Chem. Int. Ed. Engl. 2013, 52, 3350-3354), P1A1 accumulates rapidly (about 1 h) in cells to produce blue fluorescence associated with the entire cytoplasm and, to a lesser extent, the nuclear region. After 12 h, the highest fluorescence intensity is observed in the lysosomes. To track carrier-delivered P1A1 in cells, fluorescently labeled nanoparticles were generated by incorporating green-fluorescent fluorescein dye (DSPE-PEG5k-FITC) into the lipid bilayer. In cells incubated with P1A1@MSN_{LP}-COOH-LIP₁₀-FITC for 1 h, areas of colocalized blue and green fluorescence are observed on the cell surface, indicating association of intact drug-loaded nanoparticles with the cells (FIG. 7B). Unlike cells treated with carrier-free P1A1, cells exposed to P1A1-containing nanoparticles for 1 or 12 h of treatment do not show diffuse intracellular blue fluorescence in the cytoplasm. However, areas of intense blue and green fluorescence can be observed in regions also staining positive for lysosomes (see FIG. 7B). The nanoparticles appear to accumulate in acidic lysosomal vesicles of several microns in diameter, which have localized to the nuclear envelope. Closer examination of the nuclear region reveals that in a major population of cells the nanoparticles appear to have associated with the nuclear membrane to release PA payload, resulting in pan-nuclear blue fluorescence (FIG. 7C).

Again, without being bound by theory, these observations can be interpreted as follows: (i) nanocarrier-associated P1A1 enters cells and accumulates in endosomal vesicles intact; (ii) although it is likely that P1A1 is released from the carrier into the lumen of acidic, Lysotracker-stained lysosomes, no escape of payload into the cytosol is observed; (iii) once associated with the perinuclear region, the vesicles selectively release, by a yet to be determined mechanism, P1A1 into the nucleus. This type of vesicle-mediated trafficking of drug to the nucleus in lung cancer cells treated had also been shown by the inventors for platinum-(benz)acridines. However, unlike the free drugs, which also escaped from the lysosomal vesicles to produce an intensely blue stained cytoplasm and endoplasmic reticulum, P1A1@MSN_{LP}-COOH-LIP₁₀-FITC releases P1A1 into the nucleus. These results are in stark contrast to the situation observed for the delivery of doxorubicin by pH-responsive MSN carriers into cells, which results in drug release from the acidic compartments into the cytoplasm (Y. Zhang, et al., ACS Appl. Mater. Interfaces 2015, 7, 18179-18187; P. Zhang and J. Kong, Talanta 2015, 134, 501-507). Thus, the present invention possesses advantages over other studies in that the drug is released in the nucleus (instead of the cytoplasm as previous MSN studies have shown).

In summary, a highly robust nontoxic nanocarrier system was generated that is ideally suited as a potential delivery platform for PA anticancer agents. In one embodiment of the present invention, several classical design elements in a functionalized MSN material have been combined to generate a superior method of delivering PAs to the nucleus. In order to accommodate this unique payload, strategies have been developed to increase the pore dimensions of MSNs. Moreover, utilizing the special solvent system of DMF/n-decane promotes nanoparticles with these desired structural properties. After functionalization with carboxylic acid groups, nanoparticles were obtained that showed a high loading capacity for PAs (e.g., P1A1), which resulted in a material that consisted of as much as 40 wt. % payload. Finally, lipid coating, which had advantages over simple covalent PEGylation, was used to generate a chemically robust, pH-responsive, and biocompatible formulation that should prove to be effective in cancer models. P1A1@MSN_{LP}-COOH-LIP not only promises to be highly stable in circulation, but the carrier material also will likely serve as a nucleus-directed vector that delivers P1A1 directly to its pharmacological target, genomic DNA. This form of subcellular targeting should protect the reactive payload from sequestration by Pt-reactive nucleophiles in the cytosol such as glutathione (GSH), which is overexpressed in certain cancers, causing tumor resistance to platinum-based chemotherapy. Materials able to deliver a bioactive payload safely to the nucleus are of high biomedical interest (M. Murakami, et al., Sci. Translat. Med. 2011, 3, 64ra62; N. M. Sakhrani and H. Padh, Drug Des. Develop. Ther. 2013, 7, 585-599). While the triggered release of PA into the nucleus reported herein is an intriguing observation, its mechanism remains to be firmly established.

It should be understood that the present invention is not to be limited by the above description. Modifications can be made to the above without departing from the spirit and scope of the invention. It is contemplated and therefore within the scope of the present invention that any feature that is described above can be combined with any other feature that is described above. Features that are discussed as parts of compositions can be used in the disclosed methods herein and the features of the methods can be used in the compositions. Moreover, it should be understood that the present invention contemplates minor modifications that can be made to the formulations, compositions and methods of the present invention. When ranges are discussed, any number that may not be explicitly disclosed but fits within the range is contemplated as an endpoint for the range. For example, if a range of 3-20 is given, every real integer that fits within that range is contemplated as an endpoint that can be used to establish a subset range (e.g., 4, 5, 6, . . . etc. . . .19). It should be understood that features that appear in the background of the invention can be included with any feature that is disclosed that is part of the present invention. The scope of protection to be afforded is to be determined by the claims which follow and the breadth of interpretation which the law allows.

1. A pharmaceutical composition comprising a compound of formula I and a silica mesoporous nanoparticle; 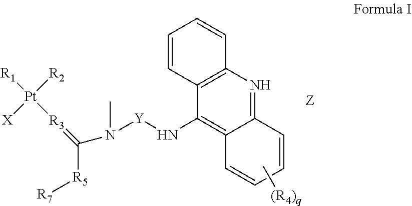
wherein X is halo, —OC(O)R₉, nitrate, H₂O or sulfate;
R₁ and R₂ are amino groups or together with the platinum atom to which they are attached, R₁ and R₂ form the ring —NH₂—(CH₂)ᵥ—NH₂— wherein v is 1, 2, or 3;
R₃ is —N(R₆)—, wherein R₆ is hydrogen or C₁-C₆alkyl;
each R₄ is independently an amino, a nitro, —NHC(O)(R₁₀), —C(O)NHR₁₀, or halo;
R₁₀ is hydrogen, C₁₋₆ alkyl, phenyl, naphthyl, C₃₋₆ cycloalkyl, norbornyl, or adamantyl;
q is 0, 1, or 2;
R₅ is a direct bond, —NH— or C₁-C₆alkylene;
or R₅ and X together with the atoms to which they are attached form a 6- or 7-membered ring, wherein said 6- or 7-membered ring contains a linking group —C(O)O— or —OC(O)—;
R₇ is hydrogen, methyl, or —C(O)O—R₈; wherein
R₈ is hydrogen, C₁₋₆ alkyl, phenyl, naphthyl, C₁₋₆ cycloalkyl, norbornyl, or adamantyl, a natural or unnatural amino acid or a peptide;
R₉ is hydrogen, C₁₋₆ alkyl, phenyl, naphthyl, C₃₋₆ cycloalkyl, norbornyl, or adamantyl;
Y is C₁-C₆alkyl; and
Z is one or more counterions sufficient to balance the charge of the compound.

2. The pharmaceutical composition of claim 1, wherein the silica mesoporous nanoparticle has a pore size of about 6.5 nm or greater.

3. The pharmaceutical composition of claim 1, wherein the silica mesoporous nanoparticle has a surface area of about at least 700 m²/g.

4. The pharmaceutical composition of claim 1, wherein the compound is [PtCl(en)(N-[acridin-9-ylaminoethyl]-N-methylpropionamidine)] dinitrate salt.

5. The pharmaceutical composition of claim 1, wherein the silica mesoporous nanoparticle is made by reacting cetyltrimethylammonium bromide with tetraethylorthosilicate.

6. The pharmaceutical composition of claim wherein the silica mesoporous nanoparticle further comprises polyethylene glycol.

7. The pharmaceutical composition of claim 1, wherein the silica mesoporous nanoparticle further comprises a lipid bilayer.

8. The pharmaceutical composition of claim 7, wherein the lipid bilayer is a phospholipid bilayer.

9. The pharmaceutical composition of claim 8, wherein the phospholipid bilayer comprises one or more of 1,2-dipalmitoyl-sn-glycero-3-phosphocholine, 1,2-dioleoyl-3-trimethylammoniumpropane, 1,2-dipalmitoyl-sn-glycero-3-phosphoethanolamine-N-[-[methoxyethyleneglycol)-2000, 1,2-Distearoyl-sn-glycero-3-phosphoethanolamine (DSPE)-conjugated and fluorescein-labeled polyethyleneglycol, and cholesterol.

10. The pharmaceutical composition of claim 1, wherein the compound is [PtCl(en)(N-[acridin-9-ylaminoethyl]-N-methylpropionamidine)] dinitrate salt and the silica mesoporous nanoparticle comprises a reaction product of cetyltrimethylammonium bromide with tetraethylorthosilicate.

11. The pharmaceutical composition of claim 10, wherein the reaction product of cetyltrimethylammonium bromide with tetraethylorthosilicate is derived from a reaction done in basic aqueous solution comprising NaOH in the presence of dimethylformamide and n-decane.

12. A method of treating cancer comprising administering to an individual in need thereof, the pharmaceutical composition of claim 1.

13. The method of claim 12, wherein the cancer is pancreatic cancer.

14. The method of claim 12, wherein the silica mesoporous nanoparticle of the pharmaceutical composition has a pore size of about 6.5 nm or greater.

15. The method of claim 12, wherein the silica mesoporous nanoparticle of the pharmaceutical composition has a surface area of about at least 700 m²/g.

16. The method of claim 12, wherein the silica mesoporous nanoparticle delivers the compound to the nucleus of a cancer cell.

17. The method of claim 12, wherein the silica mesoporous nanoparticle is charged with at least about 40 wt. % of the compound.

18. The method of claim 12, wherein the pharmaceutical composition remains intact at a pH of about 7 and the compound is released from the pharmaceutical composition at a pH of between about 4 and 5.

19. The method of claim 12, wherein the compound is [PtCl(en)(N-[acridin-9-ylaminoethyl]-N-methylpropionamidine)] dinitrate salt.

20. The method of claims 12, wherein the silica mesoporous nanoparticle is made by a process comprising reacting cetyltrimethylammonium bromide with tetraethylorthosilicate in n-decane and dimethylformamide.
